(19) United States
(12) Patent Application Publication
(10) Pub. No.: US 2009/0092853 A1
(43) Pub. Date: Apr. 9, 2009

(54) Cyclopentaphenanthrene-based compound and organic light emitting device employing the same

(76) Inventors: Sang-hoon Park, Yongin-si (KR); O-hyun Kwon, Yongin-si (KR); Dai-kyu Kim, Yongin-si (KR); Seung-gak Yang, Yongin-si (KR); Yu-jin Kim, Yongin-si (KR)

Correspondence Address: ROBERT E. BUSHNELL & LAW FIRM, 2029 K STREET NW, SUITE 600, WASHINGTON, DC, 20006-1004, US

(21) Appl. No.: 12/076,776

(22) Filed: Mar. 21, 2008

(30) Foreign Application Priority Data
Oct. 8, 2007 (KR) 10-2007-0101041

(51) Int. Cl. H05B 33/14; C07C 25/22; C07C 211/54

(52) U.S. Cl. 428/690; 570/183; 564/427

(57) ABSTRACT

Provided are a cyclopentaphenanthrene-based compound and and an organic light emitting device employing the same. The cyclopentaphenanthrene-based compound can be easily prepared, has high thermal stability and excellent hole transporting capability, and can be efficiently used to form an organic layer, particularly, a hole transport layer.

### CLAIM OF PRIORITY

This application makes reference to, incorporates the same herein, and claims all benefits accruing under 35 U.S.C.§119 from an application for CYCLOPENTAPHENANTHRENE-BASED COMPOUND AND ORGANIC LIGHT EMITTING DEVICE EMPLOYING THE SAME earlier filed in the Korean Intellectual Property Office on Oct. 8, 2007 and there duly assigned Serial No. 10-2007-0101041.

### BACKGROUND OF THE INVENTION

1. Field of the Invention

The present invention relates to a cyclopentaphenanthrene-based compound and an organic light emitting device employing the same, and more particularly, to an aromatic amine compound including cyclopentaphenanthrene and an organic light emitting device including an organic layer having the same.

2. Description of the Related Art

Organic light emitting devices are active light emitting display devices that emit light by recombination of electrons and holes in a thin layer made of a fluorescent or phosphorescent organic compound (an organic layer) when a current is applied to the organic layer. The organic light emitting devices have advantages such as lightweight, simple constitutional elements, easy fabrication process, superior image quality and wide viewing angle. Furthermore, the organic light emitting devices can accomplish perfect creation of dynamic images and high color purity. The organic light emitting devices also have electrical properties suitable for portable electronic equipment such as low power consumption and low driving voltage.

A multi-layered organic light emitting device using an aluminum quinolinol complex layer and a triphenylamine derivative layer was developed by Eastman Kodak Co. (U.S. Pat. No. 4,885,211), and a wide range of light from ultraviolet lights to infrared lights can be emitted using low-molecular weight materials when an organic emitting layer is formed (U.S. Pat. No. 5,151,629).

Light emitting devices, which are self light emitting display devices, have wide viewing angles, excellent contrast and quick response. Light emitting devices are classified into inorganic light emitting devices using inorganic compounds to form emitting layers and organic light emitting devices (OLED) using organic compounds to form emitting layers. Organic light emitting devices have higher brightness, lower driving voltages and quicker responses than inorganic light emitting devices and can realize multi colors. Thus, organic light emitting devices have been actively studied.

Typically, an organic light emitting device has an anode/organic emitting layer/cathode structure. An organic light emitting device can also have various other structures, such as an anode/hole injection layer/hole transport layer/emitting layer/electron transport layer/electron injection layer/cathode structure or an anode/hole injection layer/hole transport layer/emitting layer/hole blocking layer/electron transport layer/electron injection layer/cathode structure.

Materials that are used in organic light emitting devices can be classified into vacuum deposited materials and solution coated materials according to a method of preparing an organic layer. The vacuum deposited materials may have a vapor pressure of 10⁶ torr or greater at the temperature of 500° C. or less and be low molecular materials having a molecular weight of 1200 or less. The solution coated materials may be highly soluble in solvents to be prepared in solution phase, and include aromatic or heterocycle groups.

When an organic light emitting device is manufactured by vacuum deposition, costs may be increased due to expensive vacuum systems and high resolution pixels may not be easily manufactured if a shadow mask is used to prepare pixels for a natural color display. On the other hand, an organic light emitting device can be easily and inexpesively manufactured using solution coating such as inkjet printing, screen printing and spin coating and can have relatively high resolution compared to when using a shadow mask.

Meanwhile, when an organic light emitting device is operated or stored at a high temperature, emitting light may be changed, light emitting efficiency may be reduced, driving voltages may be increased, and lifetime may be shortened. In order to prevent those problems, a glass transition temperature (Tg) of hole injecting material, hole transporting material and emitting material should be high. In order to have high Tg, molecules of the materials have many aromatic groups, which causes crystallization of the molucules during the formation of a thin film and the crystallization may cause defects in the thin film. Meanwhile, the high Tg increases sumlimation temperature and the lifetime of organic light emitting devices may be decreased due to decomposition of materials during deposition or ununiform deposition.

Japanese Patent Publication No. hei 5-234681 discloses N,N-diphenyl-N,N-di(1-naphtyl)-1,1-biphenyl-4,4-diamine (NPD) having a higher Tg than N,N-diphenyl-N,N-dimethylphenyl-1,1-biphenyl-4,4-diamine (TPD) which has been commonly used in the art as a hole transporting material to improve thermal stability by introducing a condensed aromatic ring into a molecule.

### SUMMARY OF THE INVENTION

The present invention provides a cyclopentaphenanthrene-based compound as an organic layer forming material and an organic light emitting device employing the cyclopentaphenanthrene-based compound.

According to an aspect of the present invention, there is provided an organic compound represented by Formula 1 below:

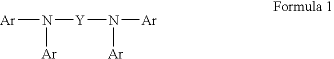

wherein Y, as a bivalent linking group, is a substituted or unsubstituted C6-C30 arylene group or a substituted or unsubstituted C2-C30 heteroarylene group;

at least one of Ars is a substituent represented by Formula 2, and the others which are identical to or different from each other are a substituted or unsubstituted C6-C30 aryl group:

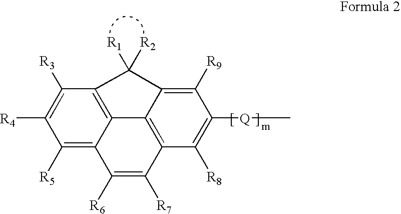

wherein R₁ and R₂ are each independently selected from the group consisting of a hydrogen atom, a halogen atom, a cyano group, a hydroxyl group, a substituted or unsubstituted C1-C20 alkyl group, a substituted or unsubstituted C3-C20 cycloalkyl group, a substituted or unsubstituted C5-C30 heterocycloalkyl group, a substituted or unsubstituted C1-C20 alkoxy group, a substituted or unsubstituted C6-C30 aryl group, a substituted or unsubstituted C6-C30 aralkyl group and a substituted or unsubstituted C2-C30 heteroaryl group; or

R₁ and R₂ are linked to form one selected from the group consisting of a substituted or unsubstituted C3-C20 aliphatic ring, a substituted or unsubstituted C5-C30 heteroaliphatic ring, a substituted or unsubstituted C6-C30 aromatic ring and a substituted or unsubstituted C2-C30 heteroaromatic ring;

R₃ to R₉ are each independently selected from the group consisting of a hydrogen atom, a halogen atom, a cyano group, a hydroxyl group, a substituted or unsubstituted C1-C20 alkyl group, a substituted or unsubstituted C3-C20 cycloalkyl group, a substituted or unsubstituted C5-C30 heterocycloalkyl group, a substituted or unsubstituted C1-C20 alkoxy group, a substituted or unsubstituted C6-C30 aryl group, a substituted or unsubstituted C6-C30 aralkyl group and a substituted or unsubstituted C2-C30 heteroaryl group;

m is an integer of 0 to 2; and

Q is a bivalent substituent represented by any one of compounds below:

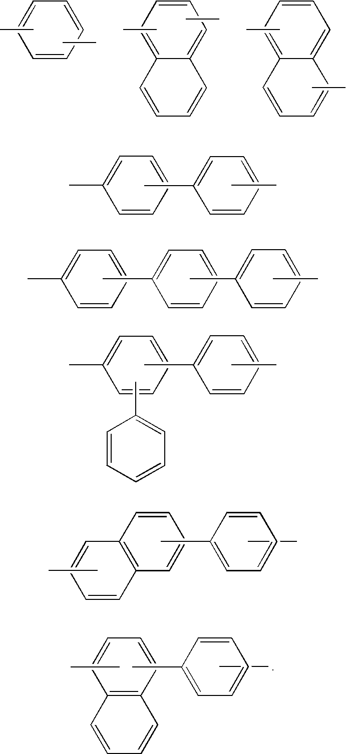

When R₁ and R₂ are linked to form a ring in Formula 2, the compound represented by Formula 2 is represented by any one of the Formulae 3 to 6 below:

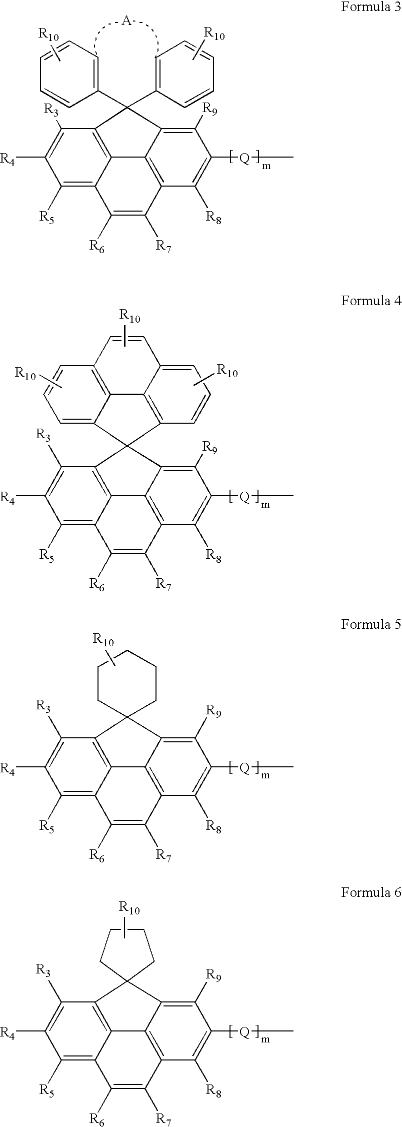

wherein R₁₀ is selected from the group consisting of a hydrogen atom, a halogen atom, a cyano group, a hydroxyl group, a substituted or unsubstituted C1-C20 alkyl group, a substituted or unsubstituted C3-C20 cycloalkyl group, a substituted or unsubstituted C5-C30 heterocycloalkyl group, a substituted or unsubstituted C1-C20 alkoxy group, a substituted or unsubstituted C6-C30 aryl group, a substituted or unsubstituted C6-C30 aralkyl group and a substituted or unsubstituted C2-C30 heteroaryl group;

A is an oxygen atom, a sulfur atom or —(CH₂)ₚ—, with the proviso that p is an integer of 1 to 5; and

R₃ to R₉, Q and m are described above.

Y is a bivalent linking group represented by any one of the compounds represented by formulae below:

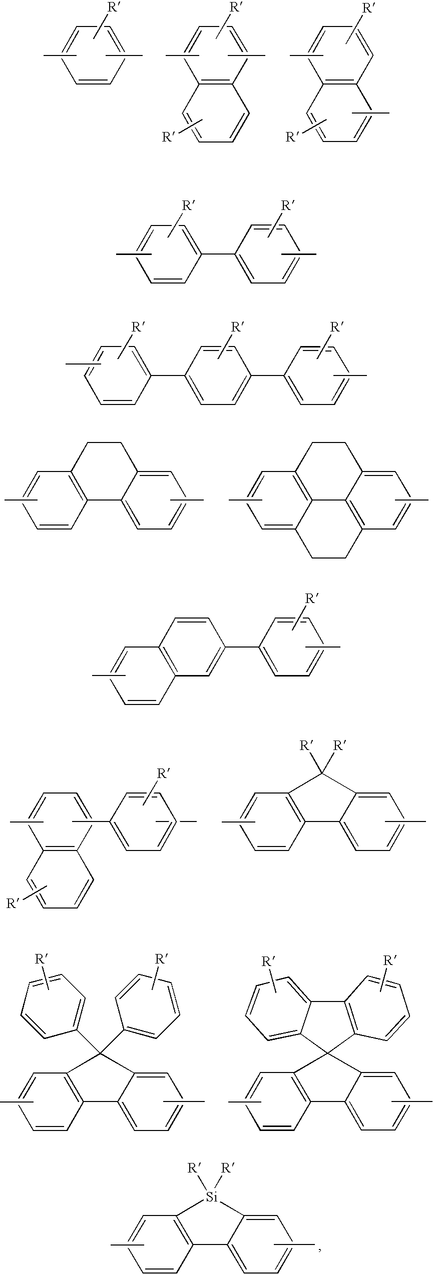

wherein R′ is selected from the group consisting of a hydrogen atom, a halogen atom, a cyano group, a hydroxyl group, a substituted or unsubstituted C1-C20 alkyl group, a substituted or unsubstituted C3-C20 cycloalkyl group, a substituted or unsubstituted C5-C30 heterocycloalkyl group and a substituted or unsubstituted C1-C20 alkoxy group.

The compound represented by Formula 2 may be represented by any one of the compounds represented by Formulae 7 to 9:

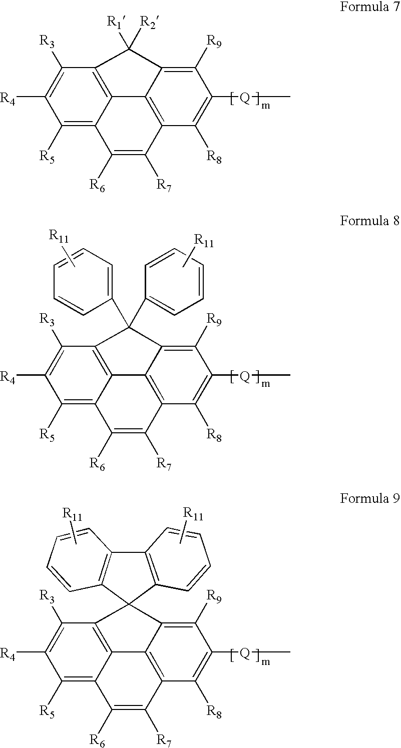

wherein R₁′, R₂′ and R₁₁ are each independently selected from the group consisting of a hydrogen atom, a halogen atom, a cyano group, a hydroxyl group, a substituted or unsubstituted C1-C20 alkyl group, a substituted or unsubstituted C3-C20 cycloalkyl group, a substituted or unsubstituted C5-C30 heterocycloalkyl group, a substituted or unsubstituted C1-C20 alkoxy group, a substituted or unsubstituted C6-C30 aryl group, a substituted or unsubstituted C6-C30 aralkyl group and a substituted or unsubstituted C2-C30 heteroaryl group; and

R₃ to R₉, Q and m are described above.

At least one of Ars may be a substituent represented by Formula 2, and the others which are identical to or different from each other may be any one of the compounds represented by formulae below:

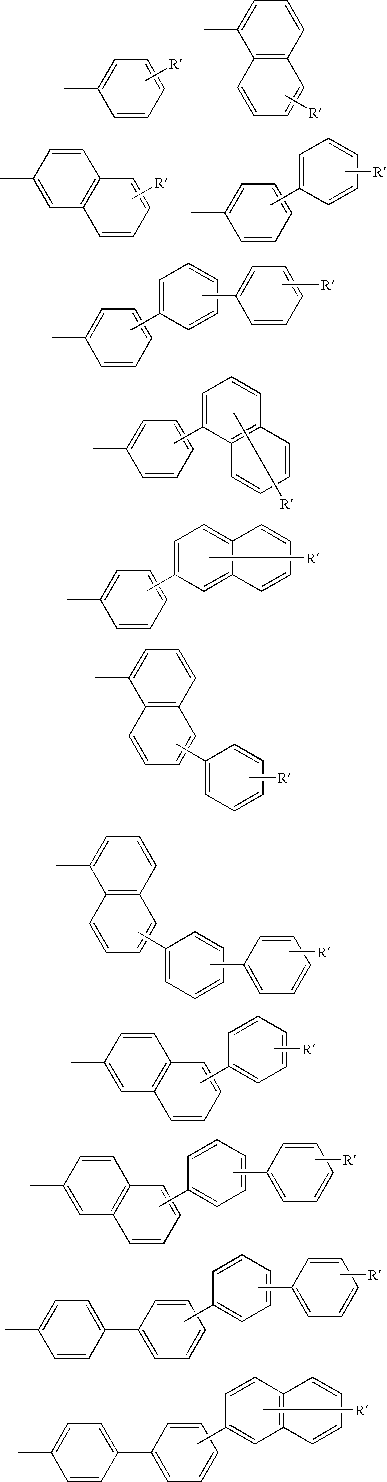

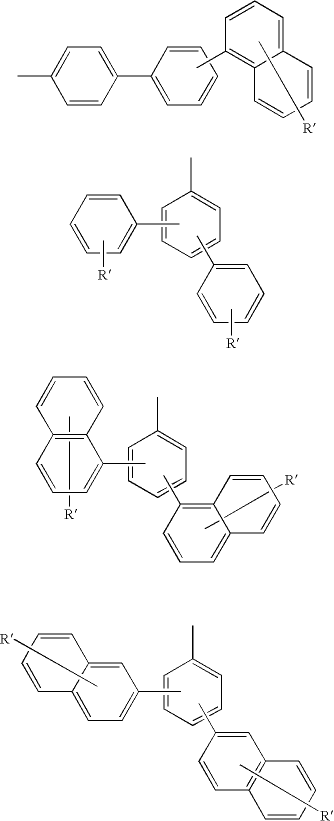

wherein R′ is selected from the group consisting of a hydrogen atom, a halogen atom, a cyano group, a hydroxyl group, a substituted or unsubstituted C1-C20 alkyl group, a substituted or unsubstituted C3-C20 cycloalkyl group, a substituted or unsubstituted C5-C30 heterocycloalkyl group and a substituted or unsubstituted C1-C20 alkoxy group.

In Formula 2, m may be an integer of 0 or 1.

According to another aspect of the present invention, there is provided an organic light emitting device comprising:

a first electrode first electrode;

a second electrode; and

at least one organic layer between the first electrode and the second electrode,

wherein the organic layer comprises the compound.

The organic layer may be an emitting layer, a hole injection layer or a hole transport layer.

The organic light emitting device may further include at least one layer selected from the group consisting of an emitting layer, a hole injection layer, a hole transport layer, an electron blocking layer, a hole blocking layer, an electron transport layer and an electron injection layer between the first electrode and the second electrode.

### BRIEF DESCRIPTION OF THE DRAWINGS

A more complete appreciation of the invention, and many of the attendant advantages thereof, will be readily apparent as the same becomes better understood by reference to the following detailed description when considered in conjunction with the accompanying drawings in which like reference symbols indicate the same or similar components, wherein:

FIG. 1A is a schematic sectional view of an organic light emitting device according to an embodiment of the present invention;

FIG. 1B is a schematic sectional view of an organic light emitting device according to another embodiment of the present invention;

FIG. 2 is a graph illustrating liquid chromatography-mass spectrometry (LC-MS) results of a compound prepared according to Example 1;

FIG. 3 is a graph illustrating thermogravimetric analysis (TGA) results of a compound prepared according to Example 1; and

FIG. 4 is a graph illustrating differential scanning calorimetry (DSC) of a compound prepared according to Example 1.

### DETAILED DESCRIPTION OF THE INVENTION

Hereinafter, the present invention will now be described more fully with reference to the accompanying drawings, in which exemplary embodiments of the invention are shown.

An aromatic amine compound including cyclopentaphenanthrene according to an embodiment of the present invention is represented by Formula 1 below.

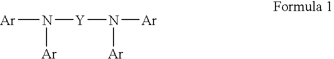

Here, Y, as a bivalent linking group, is a substituted or unsubstituted C6-C30 arylene group or a substituted or unsubstituted C2-C30 heteroarylene group;

at least one of Ars is a substituent represented by Formula 2, and the others are a substituted or unsubstituted C6-C30 aryl group:

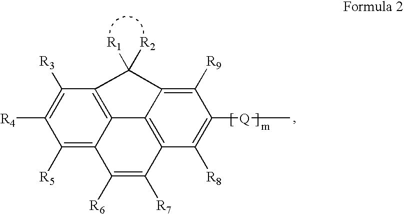

wherein m is an integer of 0 to 2.

The compound according to an embodiment of the present invention includes at least one cyclopentaphenanthrene having a rigid structure as shown in Formula 2. Meanwhile, surprisingly in the compound according to the present invention, cyclopentaphenanthrene is directly or indirectly connected to a nitrogen atom of an amino group via the C2 position of the cyclopentaphenanthrene. A variety of substituents may be linked to cyclopentaphenanthrene by introducing a halide group to the C2 position of cyclopentaphenanthrene using a method which has not been reported in the prior art. Here, cyclopentaphenanthrene may be directly connected to the nitrogen atom, and also be connected to the nitrogen atom via one or two adjacent bivalent linking group Q as shown in Formula 2. The bivalent linking group Q may be any one of the compounds represented by formulae below.

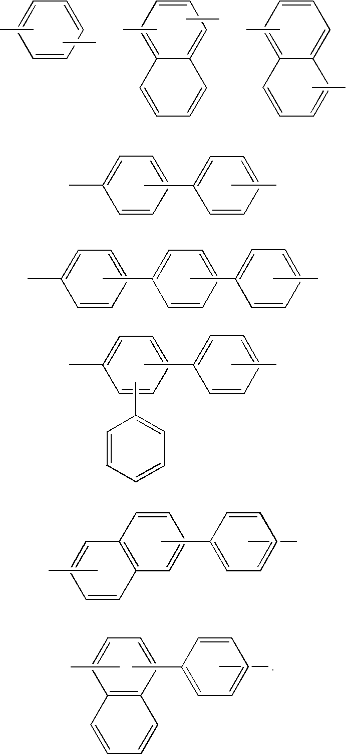

At least one hydrogen atom of the aryl group of the cyclopentaphenanthrene may be, each independently, substituted with a halogen group, a cyano group, a hydroxyl group, a substituted or unsubstituted C1-C20 alkyl group, a substituted or unsubstituted C3-C20 cycloalkyl group, a substituted or unsubstituted C5-C30 heterocycloalkyl group, a substituted or unsubstituted C1-C20 alkoxy group, a substituted or unsubstituted C6-C30 aryl group, a substituted or unsubstituted C6-C30 aralkyl group or a substituted or unsubstituted C2-C30 heteroaryl group.

Various substituents may be easily introduced into the C4 position of cyclopentaphenanthrene. That is, two hydrogen atoms at the C4 position of the cyclopentaphenanthrene may be each independently a halogen group, a cyano group, a hydroxyl group, a substituted or unsubstituted C1-C20 alkyl group, a substituted or unsubstituted C3-C20 cycloalkyl group, a substituted or unsubstituted C5-C30 heterocycloalkyl group, a substituted or unsubstituted C1-C20 alkoxy group, a substituted or unsubstituted C6-C30 aryl group, a substituted or unsubstituted C6-C30 aralkyl group or a substituted or unsubstituted C2-C30 heteroaryl group.

Meanwhile, in Formula 2, R₁ and R₂ of the C4 position of the cyclopentaphenanthrene may be linked to form a ring which may be selected from the group consisting of a substituted or unsubstituted C3-C20 aliphatic ring, a substituted or unsubstituted C5-C30 heteroaliphatic ring, a substituted or unsubstituted C6-C30 aromatic ring and a substituted or unsubstituted C2-C30 heteroaromatic ring. When the R₁ and R₂ are linked to form a ring, the compound of Formula 2 may have one of the structures shown below.

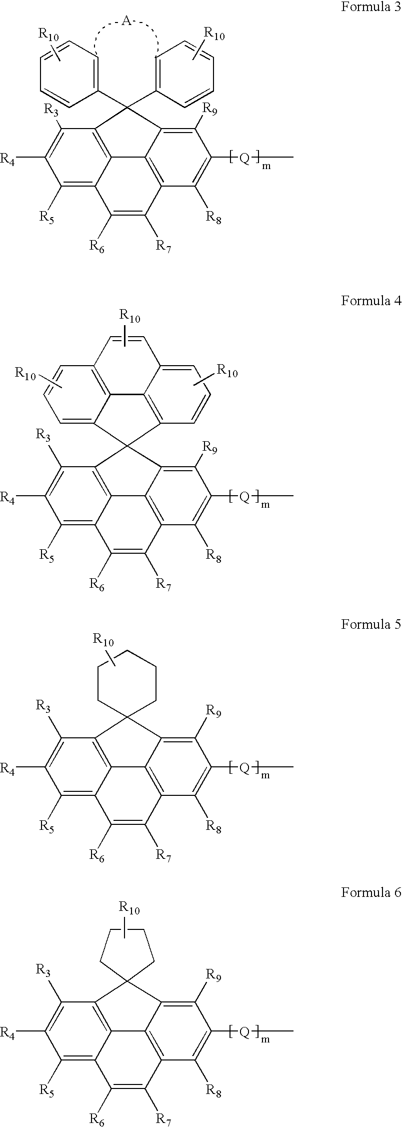

Here, R₁₀ is selected from the group consisting of a hydrogen atom, a halogen atom, a cyano group, a hydroxyl group, a substituted or unsubstituted C1-C20 alkyl group, a substituted or unsubstituted C3-C20 cycloalkyl group, a substituted or unsubstituted C5-C30 heterocycloalkyl group, a substituted or unsubstituted C1-C20 alkoxy group, a substituted or unsubstituted C6-C30 aryl group, a substituted or unsubstituted C6-C30 aralkyl group and a substituted or unsubstituted C2-C30 heteroaryl group; and

A is an oxygen atom, a sulfur atom, or —(CH₂)ₚ—, p is an integer of 1 to 5, and preferably A is a C1-C2 alkylene group; and

R₃ to R₉, Q and m are described above with respect to Formula 1.

In Formula 1, two nitrogen atoms are connected to each other by Y which is a bivalent linking group selected from the group consisting of a substituted or unsubstituted C6-C30 arylene group and a substituted or unsubstituted C2-C30 heteroarylene group, and Y is represented by any one of the formulae below:

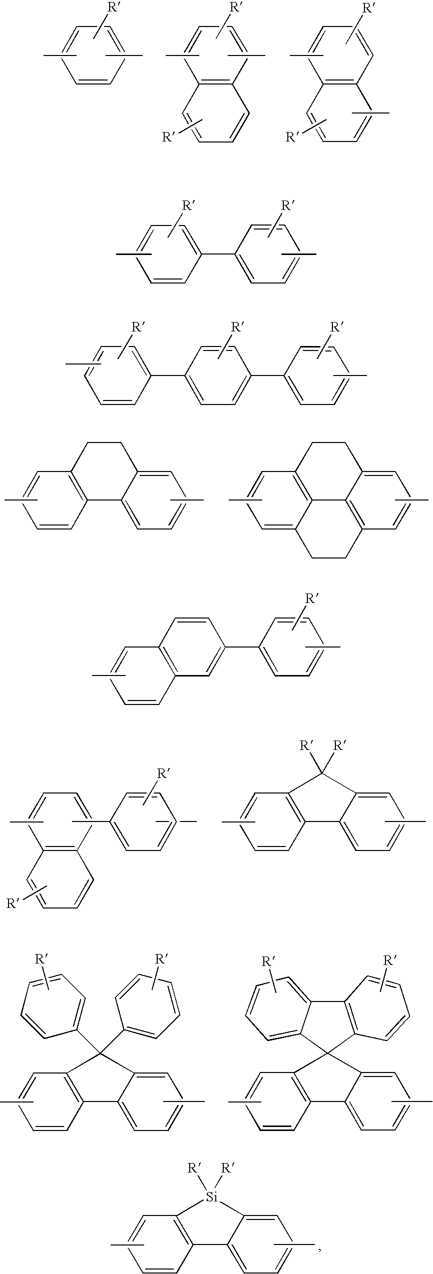

wherein R′ is a hydrogen atom, a halogen atom, a cyano group, a hydroxyl group, a substituted or unsubstituted C1-C20 alkyl group, a substituted or unsubstituted C3-C20 cycloalkyl group, a substituted or unsubstituted C5-C30 heterocycloalkyl group or a substituted or unsubstituted C1-C20 alkoxy group.

The compound of Formula 2 may be one of the compounds represented by Formulae 7 to 11 below:

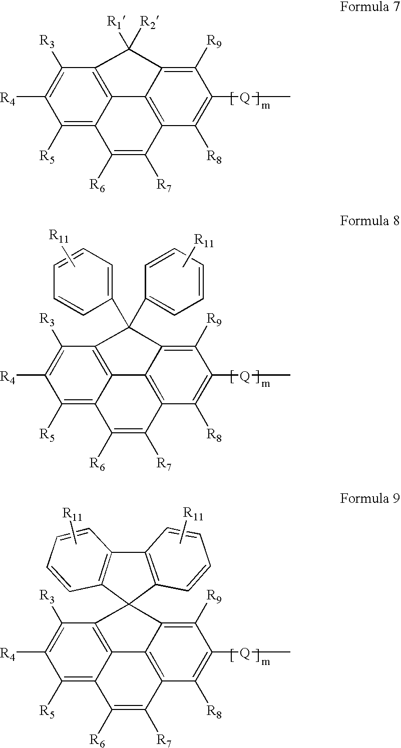

wherein R₁′, R₂′ and R₁₁ are each independently selected from the group consisting of a hydrogen atom, a halogen atom, a cyano group, a hydroxyl group, a substituted or unsubstituted C1-C20 alkyl group, a substituted or unsubstituted C3-C20 cycloalkyl group, a substituted or unsubstituted C5-C30 heterocycloalkyl group, a substituted or unsubstituted C1-C20 alkoxy group, a substituted or unsubstituted C6-C30 aryl group, a substituted or unsubstituted C6-C30 aralkyl group and a substituted or unsubstituted C2-C30 heteroaryl group; and

R₃to R₈, Q and m are described above with respect to Formula 1.

In Formula 1, Ar which is not represented by Formula 2 may be a substituted or unsubstituted C6-C30 aryl group, and preferably represented by any one of the formulae below.

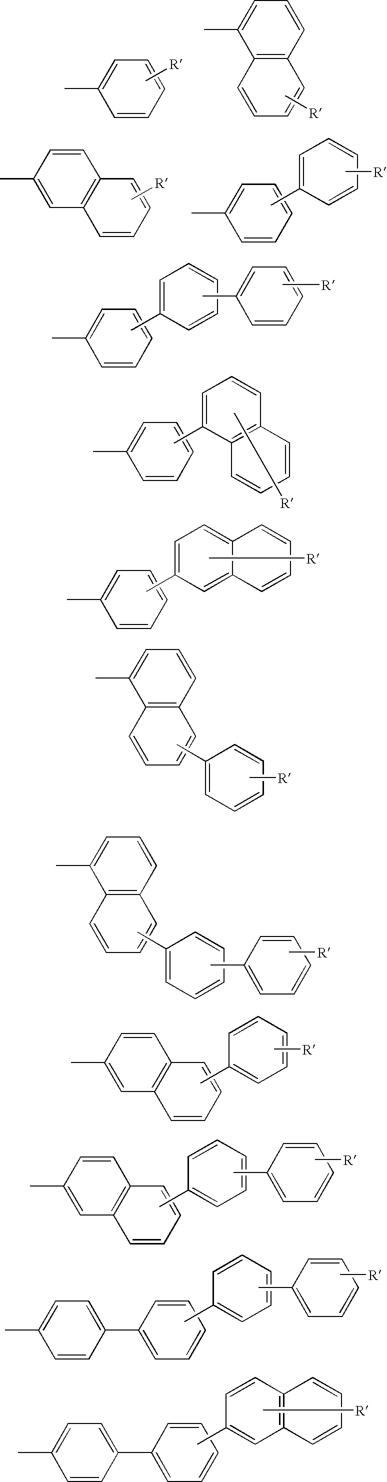

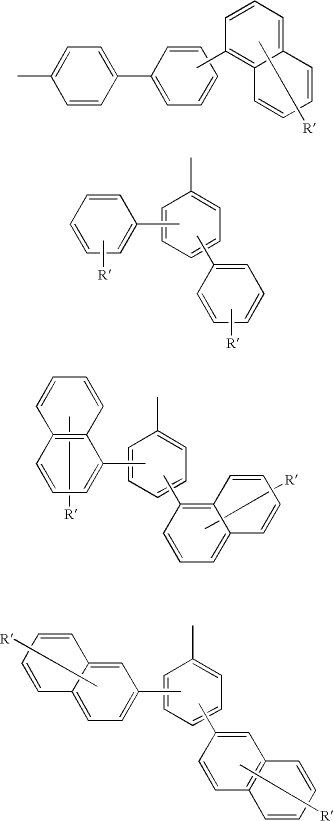

In the formulae described above, R′ is selected from the group consisting of a hydrogen atom, a halogen atom, a cyano group, a hydroxyl group, a substituted or unsubstituted C1-C20 alkyl group, a substituted or unsubstituted C3-C20 cycloalkyl group, a substituted or unsubstituted C5-C30 heterocycloalkyl group and a substituted or unsubstituted C1-C20 alkoxy group.

In the formulae described above, the aryl group is a monovalent group having two or more aromatic rings which can be bound to be fused with each other. The heteroaryl group is an aryl group a group at least one carbon atom of which is substituted with at least one of N, O, S and P.

Meanwhile, the cycloalkyl group is an alkyl group having a ring and the heterocycloalkyl group is a cycloalkyl at least one carbon atom of which is substituted with at least one of N, O, S and P.

When the alkyl group, the alkoxy group, the aryl group, the heteroaryl group, the cycloalkyl group and the heterocycloalkyl group are substituted, the substitutents may be at least one of —F; —Cl; —Br; —CN; —NO₂; —OH; a C1-C20 alkyl group that is unsubstituted or substituted with —F, —Cl, —Br, —CN, —NO₂ or —OH; a C1-C20 alkoxy group that is unsubstituted or substituted with —F, —Cl, —Br, —CN, —NO₂ or —OH; a C6-C30 aryl group that is unsubstituted or substituted with a C1-C20 alkyl group, a C1-C20 alkoxy group, —F, —Cl, —Br, —CN, —NO₂ or —OH; a C2-C30 heteroaryl group that is unsubstituted or substituted with a C1-C20 alkyl group, a C1-C20 alkoxy group, —F, —Cl, —Br, —CN, —NO₂ or —OH; a C5-C20 cycloalkyl group that is unsubstituted or substituted with a C1-C20 alkyl group, a C1-C20 alkoxy group, —F, —Cl, —Br, —CN, —NO₂ or —OH; a C5-C30 heterocycloalkyl group that is unsubstituted or substituted with a C1-C20 alkyl group, a C1-C20 alkoxy group, —F, —Cl, —Br, —CN, —NO₂ or —OH; and —N(G6)(G7). Here, G6 and G7 are each independently a hydrogen atom; C1-C10 alkyl group; or a C6-C30 aryl group substituted with a C1-C10 alkyl group.

In more particular, R₁ to R₁₁ are each independently selected from the group consisting of a hydrogen atom, a halogen atom, a cyano group, a hydroxyl group, a C1-C10 alkyl group, a C1-C10 alkoxy group and a substituted or unsubstituted derivative as follows: a phenyl group, a biphenyl group, a pentalenyl group, an indenyl group, a naphthyl group, a biphenylenyl group, an anthracenyl group, an azulenyl group, a heptalenyl group, an acenaphtylenyl group, a phenalenyl group, a fluorenyl group, a methylanthryl group, a phenanthrenyl group, a triphenylenyl group, a pyrenyl group, a chrysenyl group, an ethyl-chrysenyl group, a picenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a fluorenyl group, a pyranthrenyl group, an ovalenyl group, a carbazolyl group, a thiophenyl group, an indolyl group, a purinyl group, a benzimidazolyl group, a quinolinyl group, a benzothiophenyl group, a parathiazinyl group, a pyrrolyl group, a pyrazolyl group, an imidazolyl group, an imidazolinyl group, an oxazolyl group, a thiazolyl group, a triazolyl group, a tetrazolyl group, an oxadiazolyl group, a pyridinyl group, a pyridazinyl group, a pyrimidinyl group, a pyrazinyl group, a thianthrenyl group, a cyclopentyl group, a cyclohexyl group, an oxyranyl group, a pyrrolidinyl group, a pyrazolidinyl group, an imidazolidinyl group, a piperidinyl group, a piperazinyl group, a morpholinyl group, a di(C6-C30 aryl)amino group, a tri(C6-C30 aryl)silyl group and derivatives thereof.

Here, the term “derivative” indicates a group in which at least one of the hydrogen groups is substituted with the substituents described above.

According to an embodiment of the present invention, a cyclopentaphenanthrene-based compound represented by Formula 1 has high solubility in a solvent in the formation of an organic layer, high thermal stability and excellent charge transporting properties.

The compound according to the present invention may be represented by one of the compounds represented by Formulae 10 to 60, but is not limited thereto.

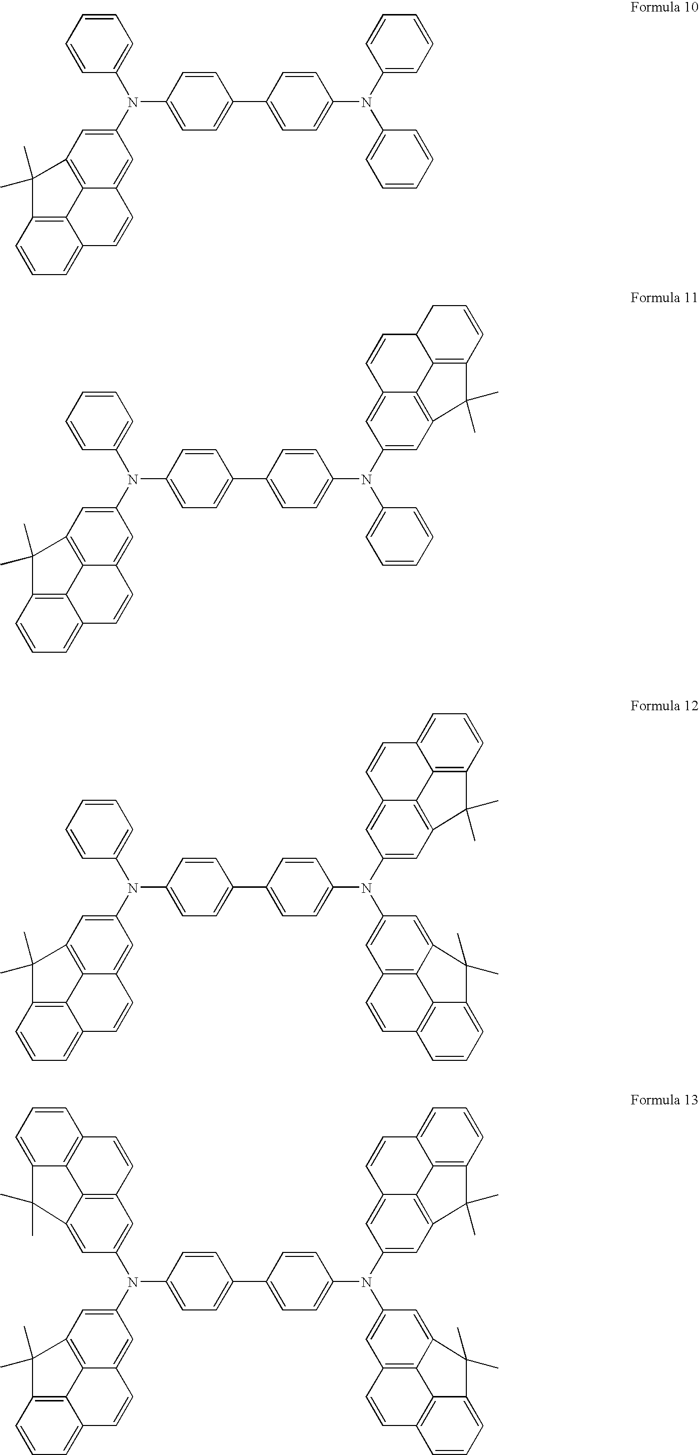

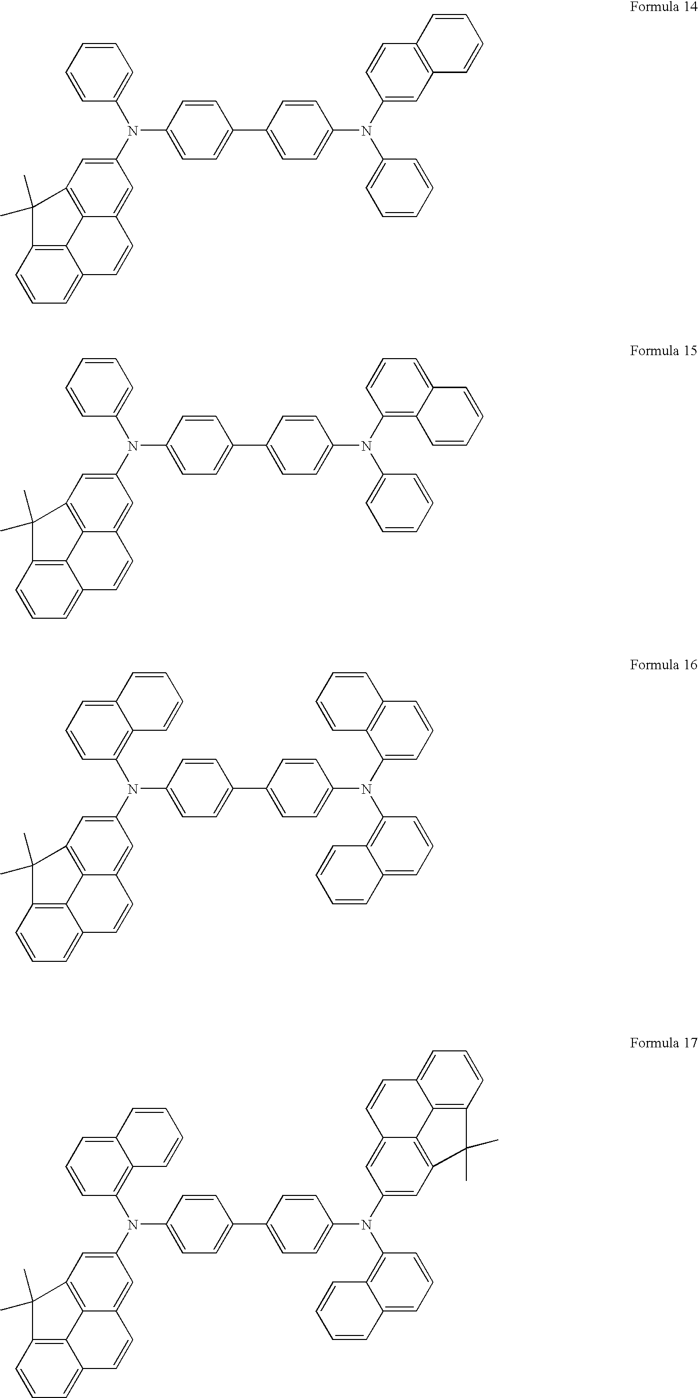

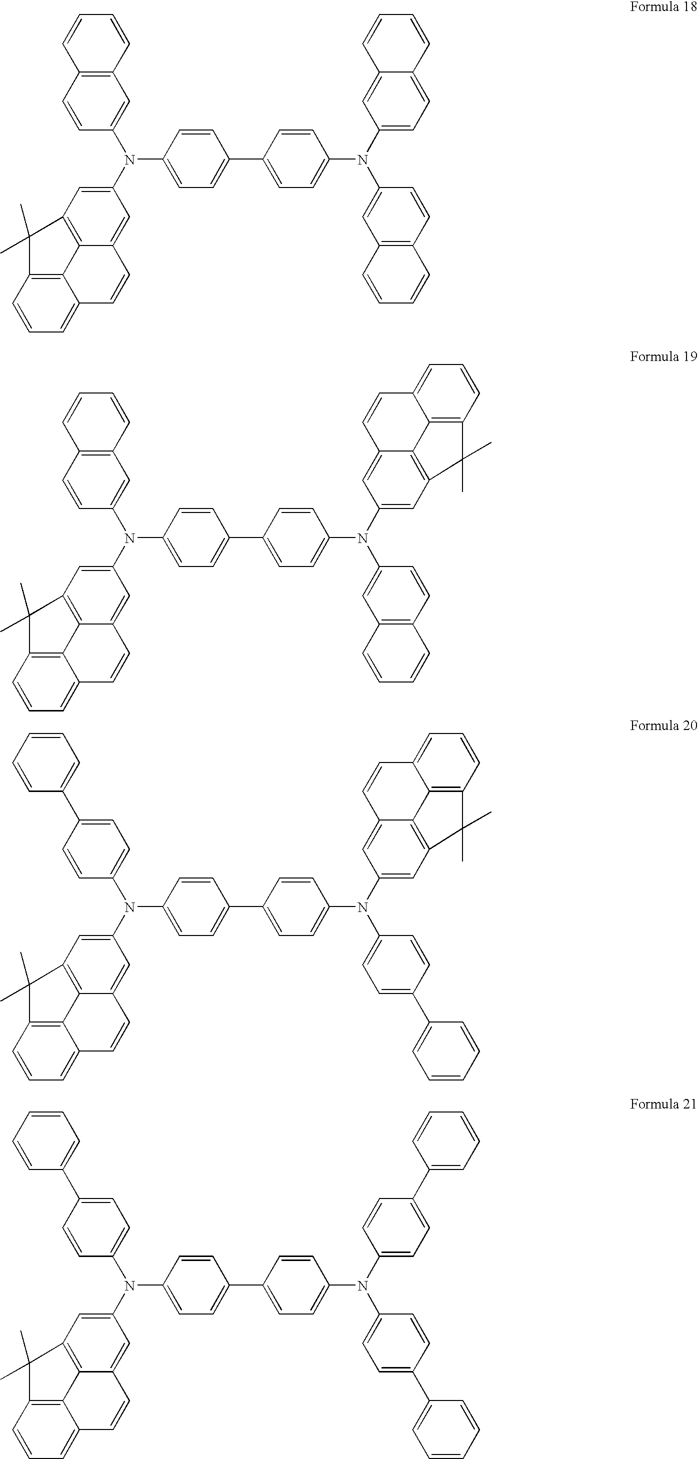

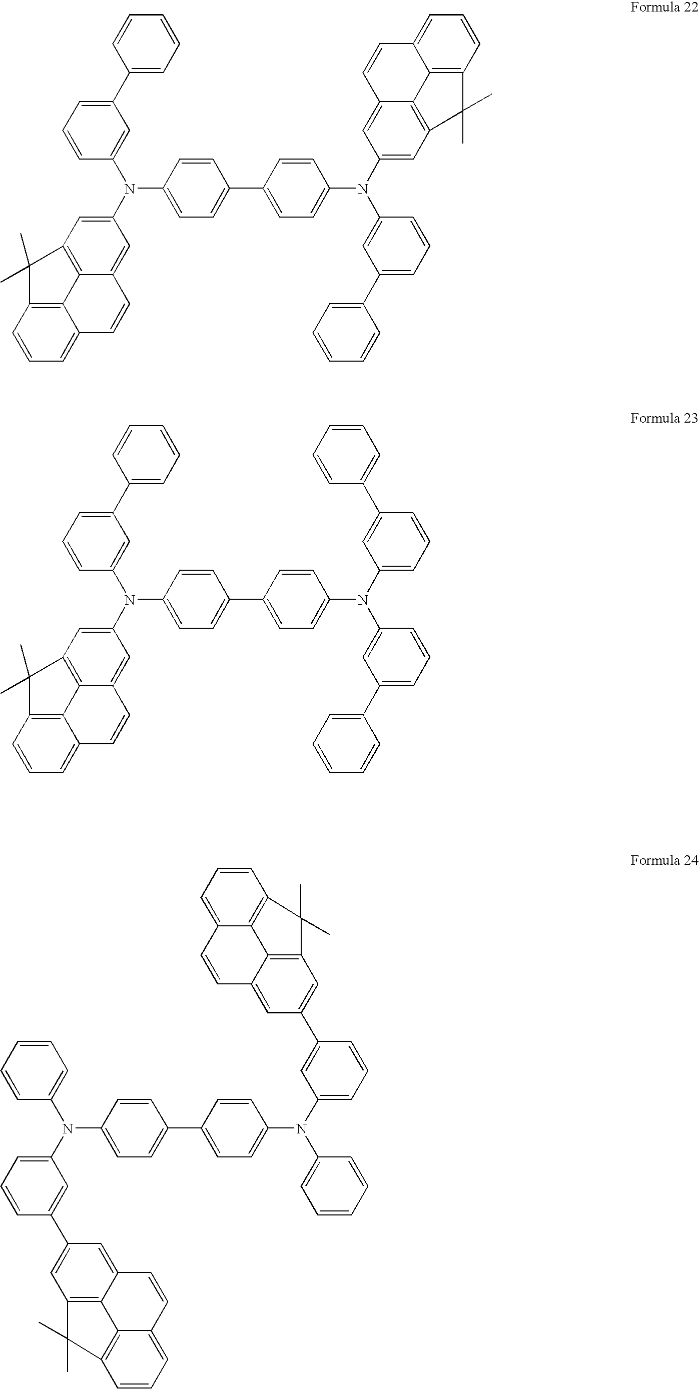

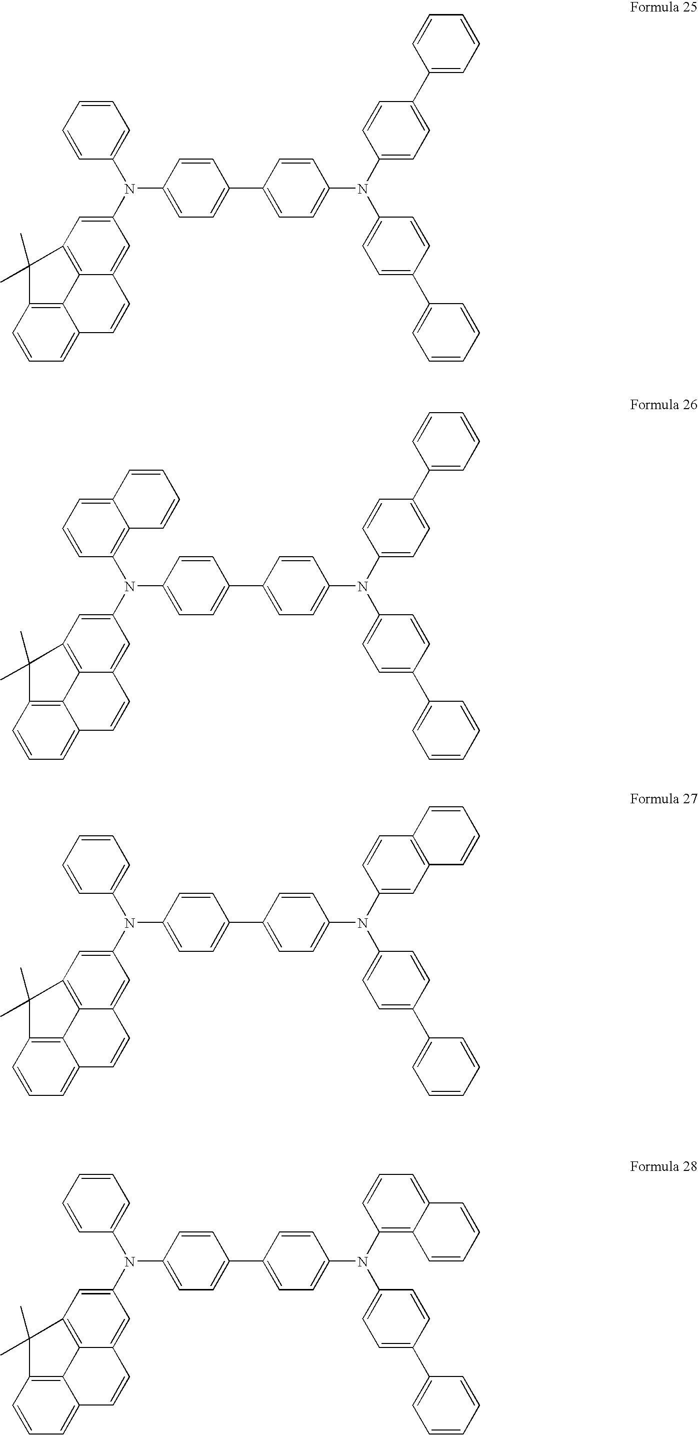

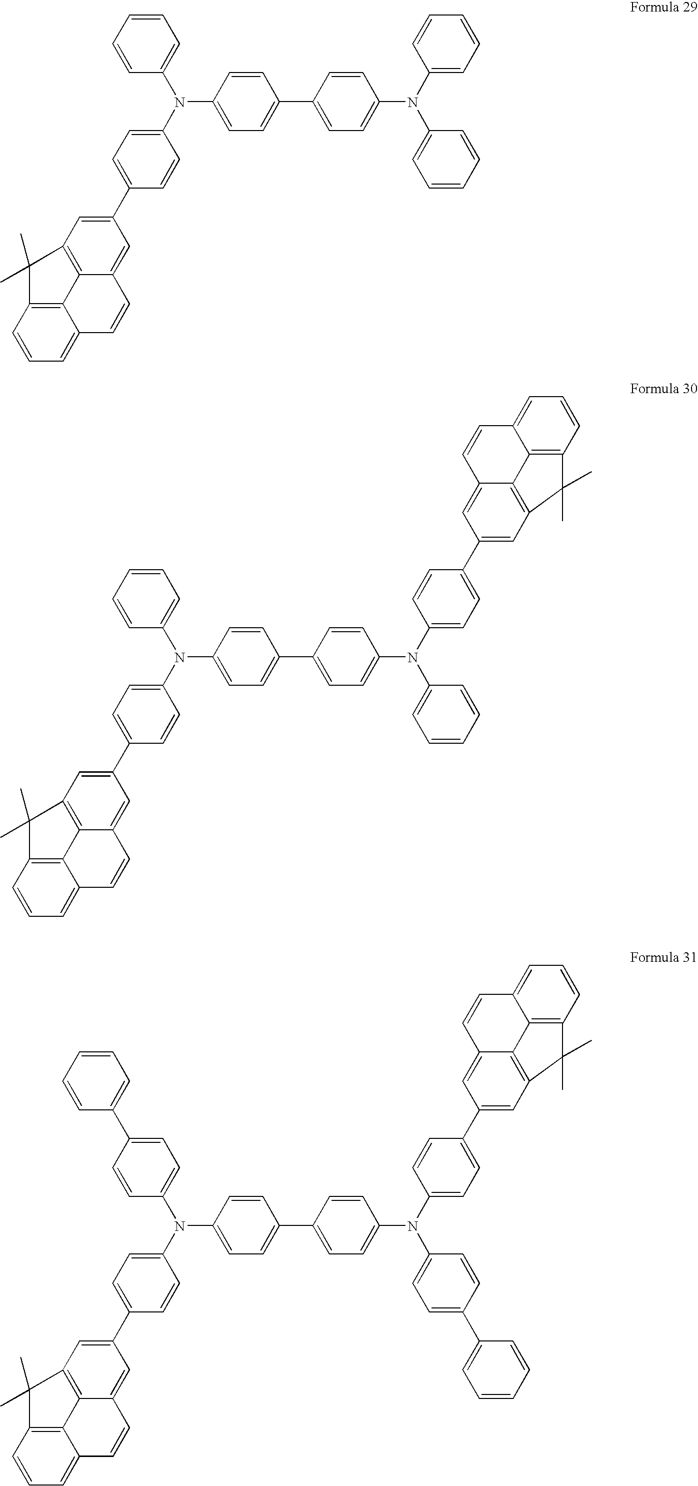

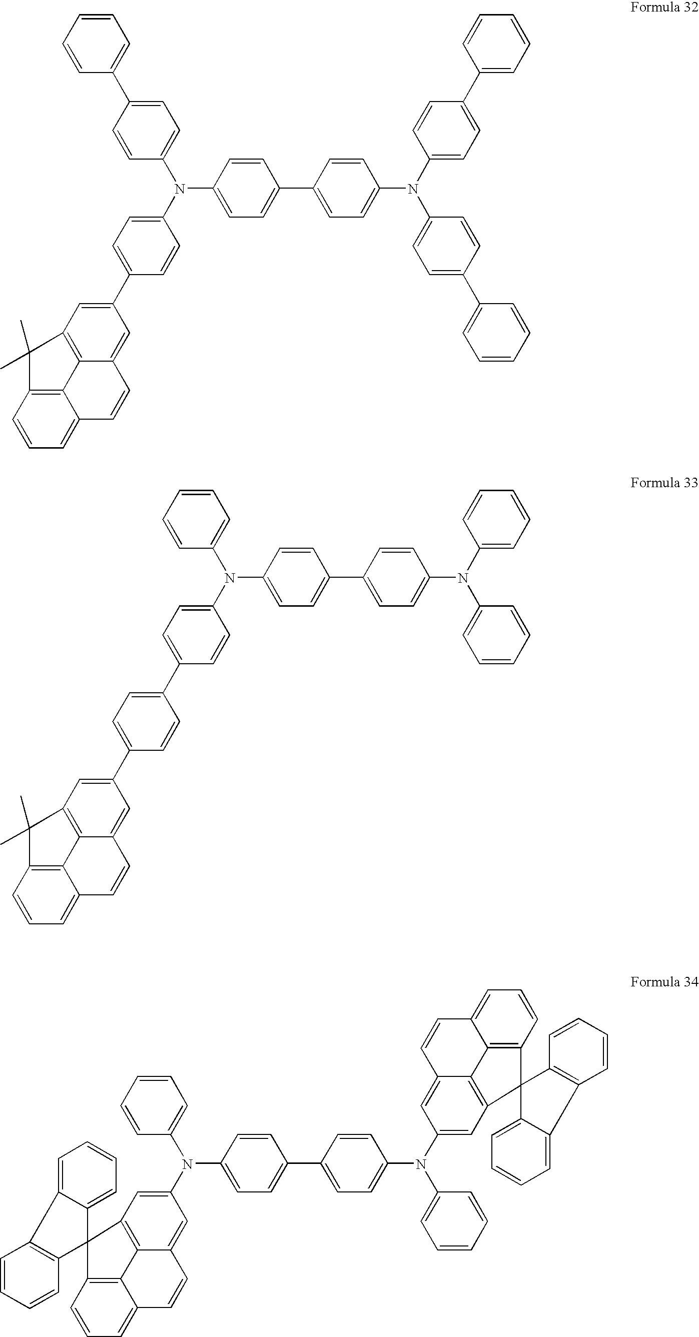

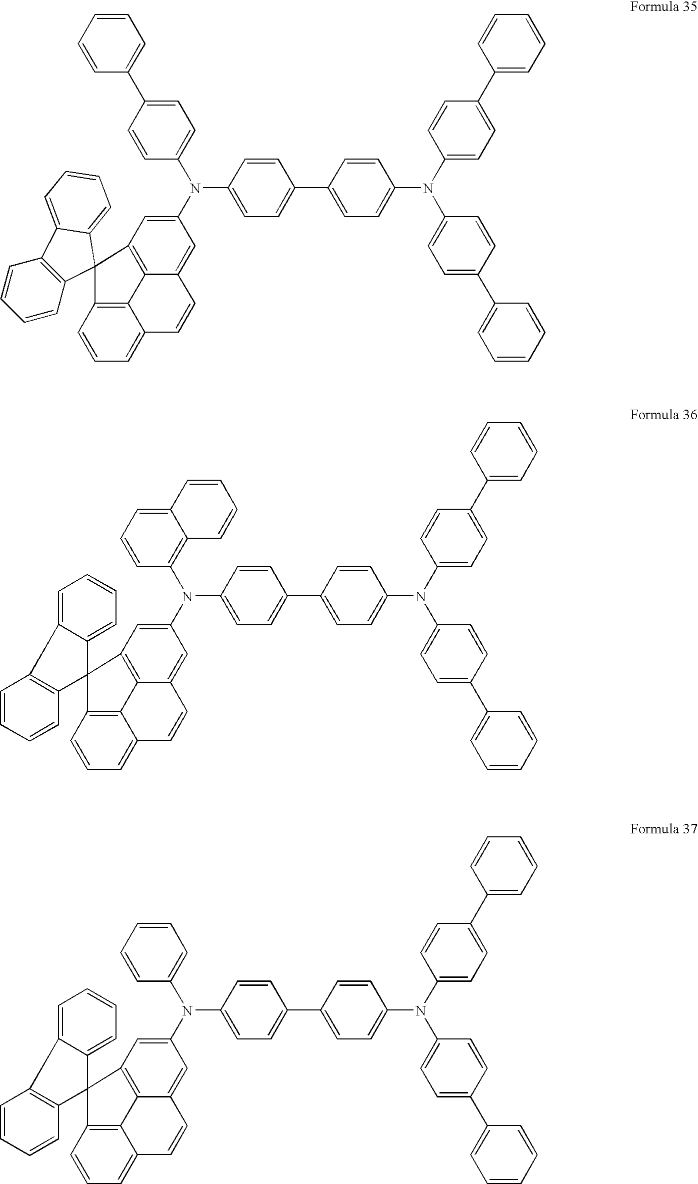

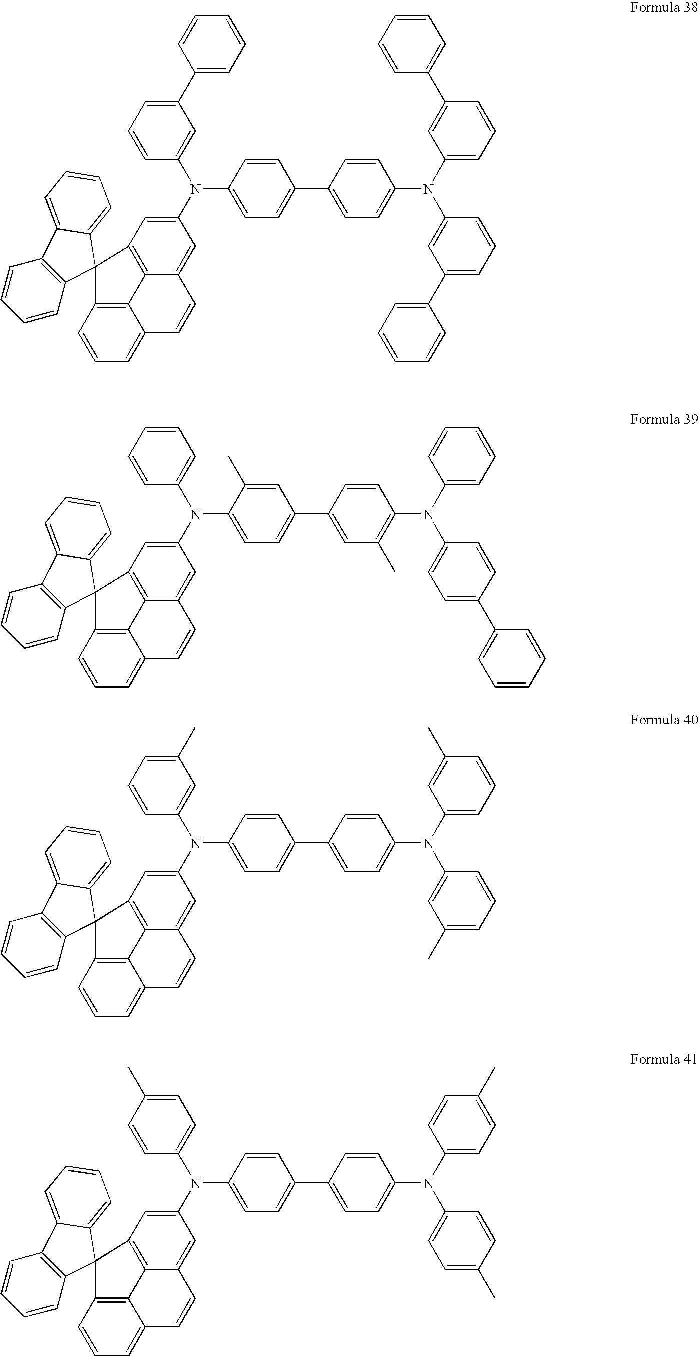

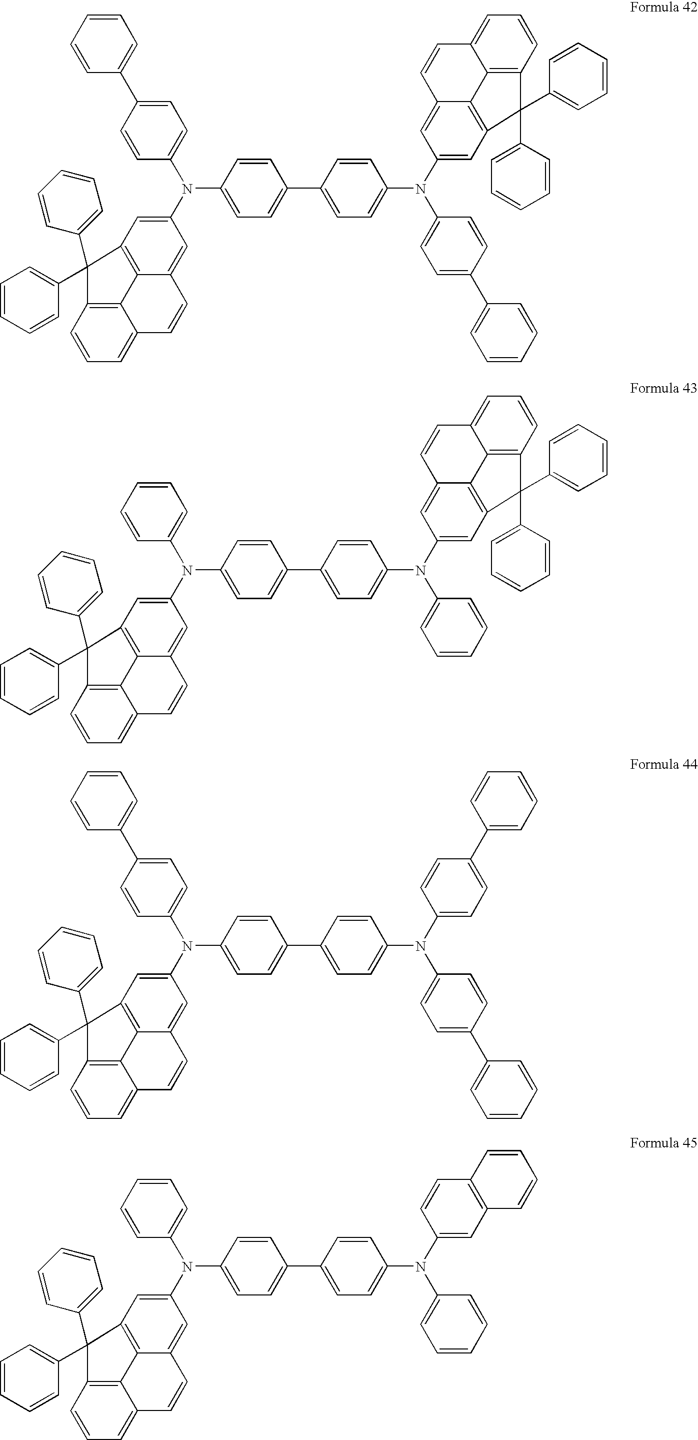

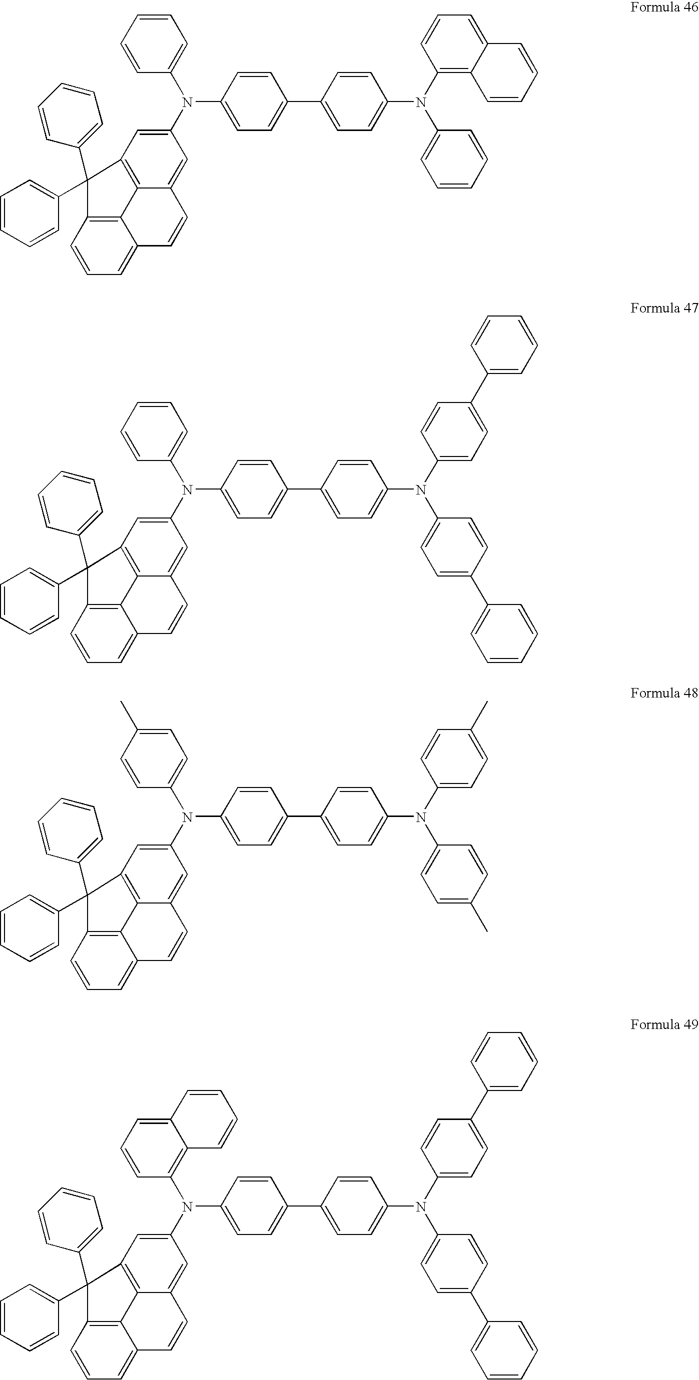

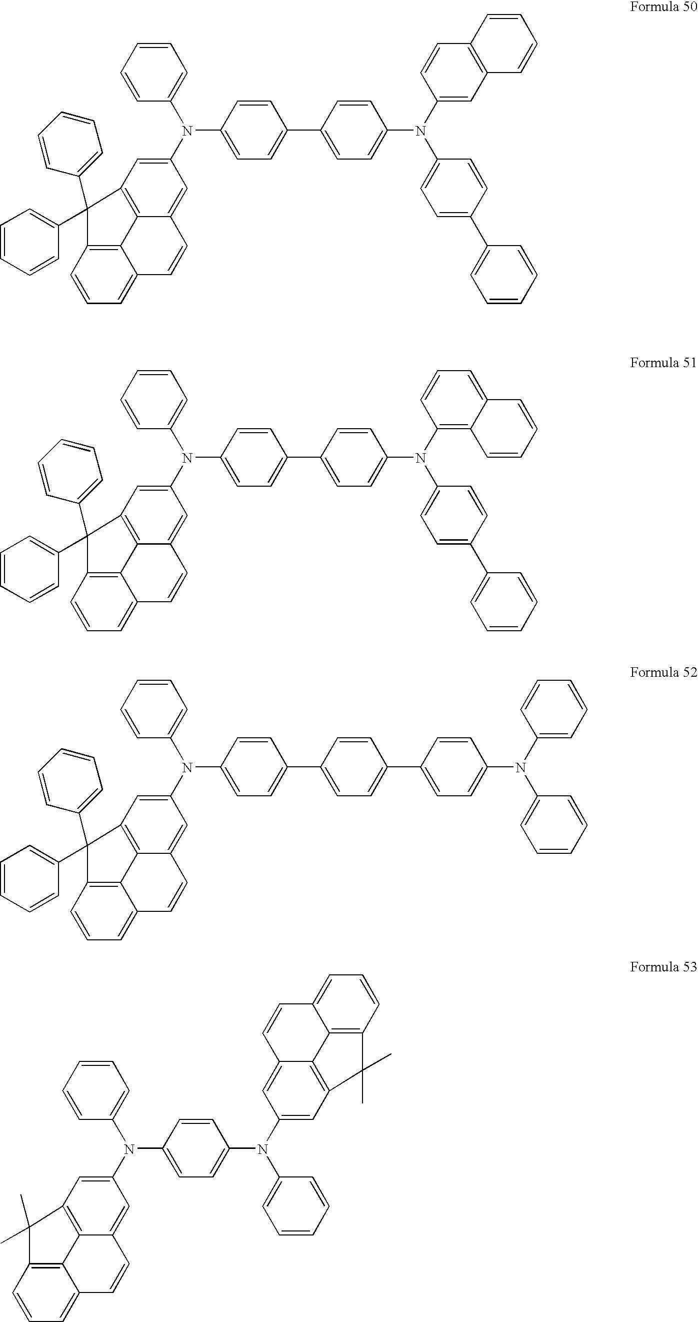

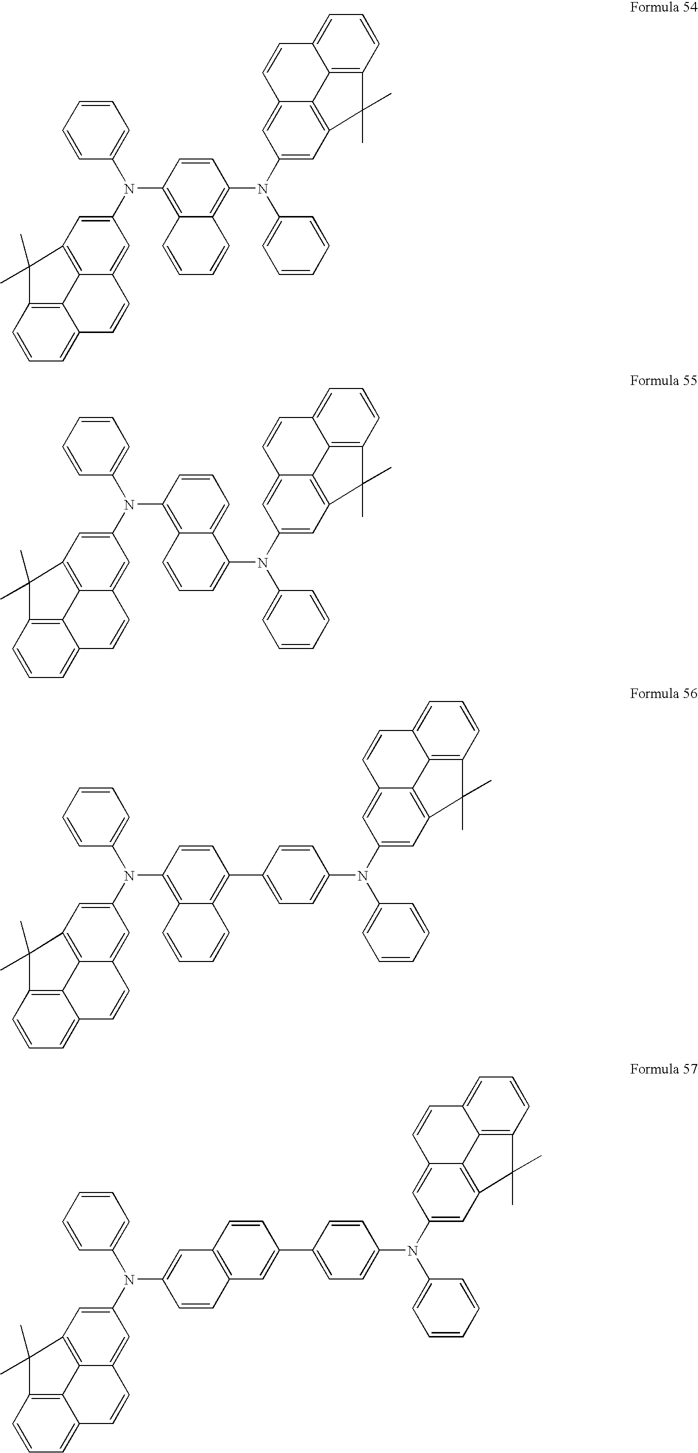

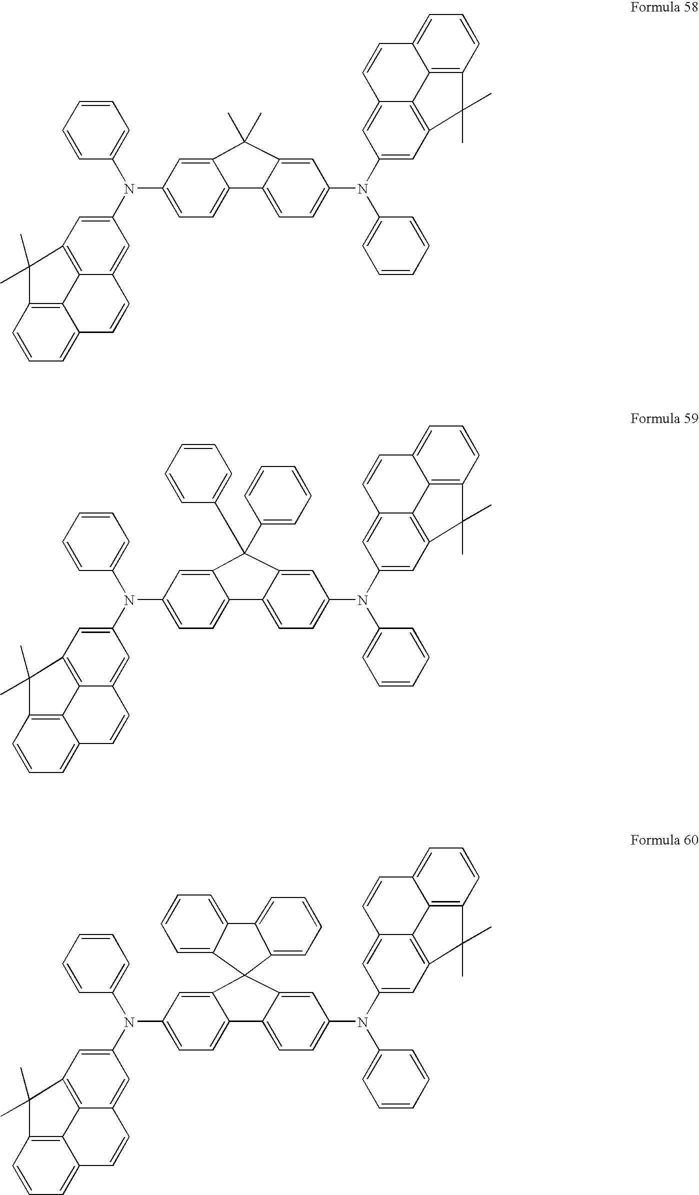

The compound represented by Formulae 1 and 2 according to the present invention may be synthesized using a method that is commonly used in the art. A synthetic pathway of the compound is described with respect to Synthesis Examples and Examples below.

An organic light emitting device according to the present invention may include a first electrode; a second electrode; and an organic layer interposed between the first electrode and the second electrode, wherein the organic layer includes at least one compound represented by Formula 1.

The compound of Formula 1 is suitably used to form an organic layer, preferably an emitting layer, a hole injection layer or a hole transport layer, and more preferably a hole transport layer, of an organic light emitting device.

The organic light emitting device of the present invention has improved emitting properties such as excellent driving voltage properties and high color purity by employing a compound having high solubility and thermal stability and capable of forming a stable organic layer when compared to a conventional organic light emitting 11 device prepared using a solution coating method and having low stability of organic layer.

The organic light emitting device of the present invention may have various structures. That is, the organic light emitting device may further include at least one layer selected from the group consisting of a hole injection layer, a hole transport layer, a hole blocking layer, an electron blockig layer, an electron transport layer and an electron injection layer between the first electrode and the second electrode.

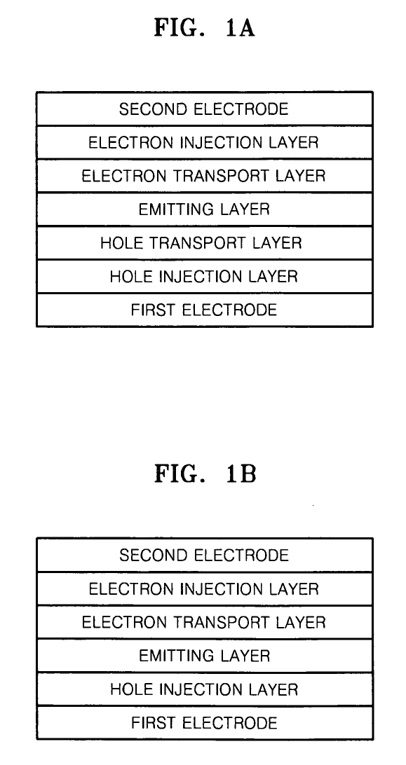

More particularly, FIGS. 1A and 1B are schematic sectional views of organic light emitting devices according to embodiments of the present invention. The organic light emitting device of FIG. 1A has a structure of a first electrode/a hole injection layer/a hole transport layer/an emitting layer/an electron transport layer/an electron injection layer/a second electrode. The organic light emitting device of FIG. 1B has a structure of a first electrode/a hole injection layer/an emitting layer/an electron transport layer/an electron injection layer/a second electrode. An emitting layer of the organic light emitting device of the present invention may include a phosphorescent or fluorescent dopant for red, green, blue or white color. The phosphorescent dopant may be an organic metal compound including at least one element selected from the group consisting of Ir, Pt, Os, Ti, Zr, Hf, Eu, Tb and Tm.

Hereinafter, a method of preparing an organic light emitting device according to the present invention will be described with reference to FIG. 1A.

First, a first electrode is formed on a substrate, for example, by depositing or sputtering a high work-function material. The first electrode can be an anode. The substrate, which can be any substrate that is used in conventional organic light emitting devices, may be a glass substrate or a transparent plastic substrate with excellent mechanical strength, thermal stability, transparency, surface smoothness, ease of treatment, and waterproof. The material that is used to form the first electrode can be ITO, IZO, SnO₂, ZnO, or any transparent material which has high conductivity.

Then, a hole injection layer (HIL) can be formed on the first electrode by vacuum deposition, spin coating, casting, langmuir Blodgett (LB), or the like.

When the hole injection layer is formed by vacuum deposition, deposition conditions may vary according to a compound that is used to form the hole injection layer, and the structure and thermal properties of the hole injection layer to be formed. In general, however, conditions for vacuum deposition may include a deposition temperature of 100 to 500° C., a pressure of 10⁻⁸ torr to 10⁻³ torr, a deposition speed of 0.01 to 100 Å/sec, and a layer thickness of 10 Å to 5 μm .

When the hole injection layer is formed by spin coating, coating conditions may vary according to a compound that is used to form the hole injection layer, and the structure and thermal properties of the hole injection layer to be formed. In general, however, conditions for spin coating may include a coating speed of 2000 to 5000 rpm and a heat-treatment temperature of about 80 to 200° C. to remove a solvent after coating.

The material that is used to form the hole injection layer may be a compound represented by Formula 1. The thickness of the HIL may be in the range of about 100 to 10000 Å, and preferably in the range of 100 Å to 1000 Å. When the thickness of the HIL is less than 100 Å, the hole injecting ability of the HIL may be reduced. On the other hand, when the thickness of the HIL is greater than 10000 Å, a driving voltage of the device may be increased.

Then, a hole transport layer (HTL) can be formed on the HIL by vacuum deposition, spin coating, casting, LB, or the like. When the HTL is formed by vacuum deposition or spin coating, the conditions for deposition and coating are similar to those for the formation of the HIL, although conditions for the deposition and coating may vary according to the material that is used to form the HTL.

The HTL can be formed of the compound of Formula 1 described above. The thickness of the HTL may be in the range of about 50 to 1000 Å, and preferably 100 to 600 Å. When the thickness of the HTL is less than 50 Å, a hole transporting ability of the HTL may be reduced. On the other hand, when the thickness of the HTL is greater than 1000 □, the driving voltage of the device may be increased.

Then, an emitting layer. (EML) can be formed on the HTL by vacuum deposition, spin coating, casting, LB, or the like. When the EML is formed by vacuum deposition or spin coating, the conditions for deposition and coating are similar to those for the formation of the HIL, although the conditions for deposition and coating may vary according to the material that is used to form the EML.

The thickness of the EML may be in the range of about 100 to 1000 Å, and preferably in the range of 200 to 600 Å. When the thickness of the EML is less than 100 Å, the emitting ability of the EML may be reduced. On the other hand, when the thickness of the EML is greater than 1000 ÅA, the driving voltage of the device may be increased.

A hole blocking layer (HBL) can be formed on the HTL by vacuum deposition, spin coating, casting, LB, or the like, to prevent diffusion of triplet excitons or holes into an electron transport layer when the phosphorescent dopant is used to form the EML. When the HBL is formed by vacuum deposition or spin coating, the conditions for deposition and coating are similar to those for the formation of the HIL, although the conditions for deposition and coating may vary according to the material that is used to form the HBL. The HBL may be formed of, for example, an oxadiazole derivative, a triazole derivative, a phenanthroline derivative, BCP or an aluminum complex.

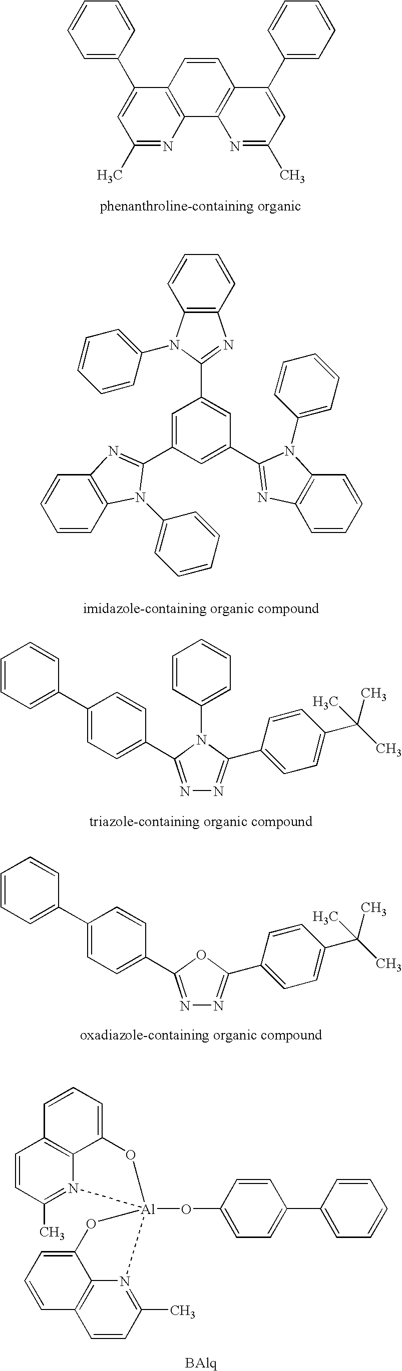

The thickness of the HBL may be in the range of about 50 to 1000 Å, and preferably in the range of 100 to 300 Å. When the thickness of the HBL is less than 50 Å, the hole blocking ability of the HBL may be reduced. On the other hand, when the thickness of the HBL is greater than 1000 Å, the driving voltage of the device may be increased.

Then, an electron transport layer (ETL) is formed by vacuum deposition, spin coating, casting, or the like. When the ETL is formed by vacuum deposition or spin coating, the conditions for deposition and coating are, in general, similar to those for the formation of the HIL, although the conditions for the deposition and coating conditions may vary according to the material that is used to form the ETL. The ETL may be formed of a known material in the art which stably transports injected electrons from a cathode, for example, an oxazole-based compound, an isooxazole-based compound, a triazole-based compound, an isothiazole-based compound, an oxadiazole-based compound, a thiadiazole-based compound, a perylene-based compound, an aluminum complex such as tris(8-quinolinolato)-aluminium (Alq3), BAlq and SAlq, Almq3, a gallium complex such as Gaq′2OPiv, Gaq′2OAc and 2(Gaq′2), or the like.

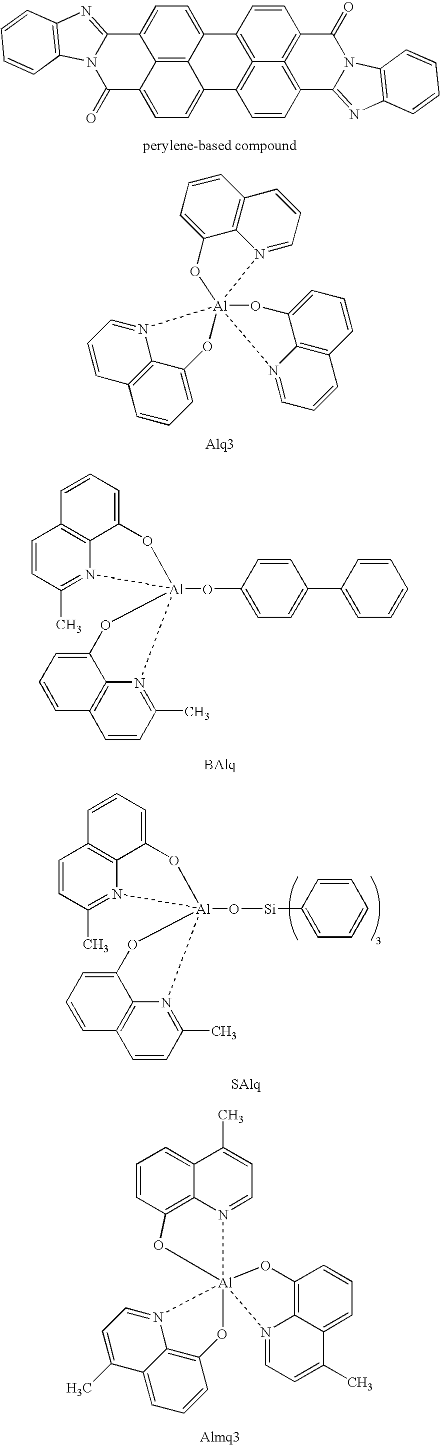

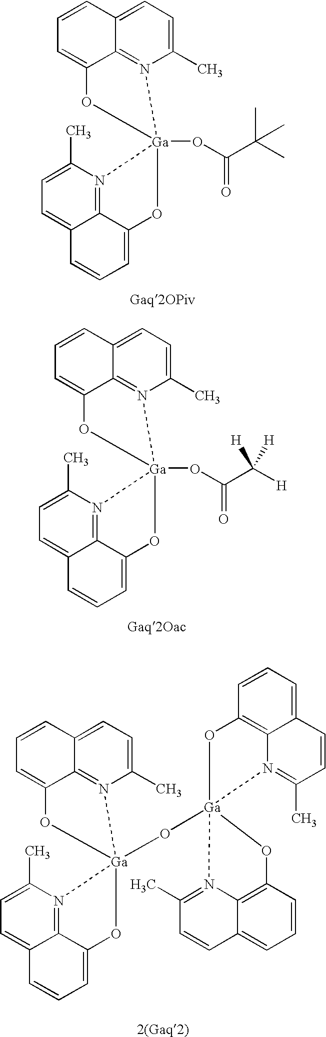

The thickness of the ETL may be in the range of about 100 to 1000 Å, and preferably 200 to 500 Å. When the thickness of the ETL is less than 100 Å, the electron transporting ability of the ETL may be reduced. On the other hand, when the thickness of the ETL is greater than 1000 Å, the driving voltage of the device may be increased.

Then, an electron injection layer (EIL), which is formed of a material allowing easy injection of electrons from a cathode, can be formed on the ETL. The material that is used to form the EIL is not limited.

The EIL may be formed of LiF, NaCl, CsF, Li₂O, BaO, or the like, which is known in the art. Conditions for the deposition of the EIL are, in general, similar to conditions for the formation of the HIL, although they may vary according to the material that is used to form the EIL.

The thickness of the EIL may be in the range of about 1 to 100 Å, and preferably 5 to 50 Å. When the thickness of the EIL is less than 1 Å, the electron injecting ability of the EIL may be reduced. On the other hand, when the thickness of the EIL is greater than 100 Å, the driving voltage of the device may be increased.

Finally, a second electrode can be formed on the EIL by vacuum deposition, sputtering, or the like. The second electrode can be used as a cathode. The second electrode may be formed of a low work-function metal, an alloy, an electrically conductive compound, or a combination of these. In detail, the second electrode may be formed of Li, Mg, Al, Al—Li, Ca, Mg—In, Mg—Ag, or the like. Alternatively, a transparent cathode formed of ITO or IZO can be used to produce a top emission light emitting device.

The organic light emitting device according to the present invention has low driving voltage, high efficiency and long lifetime.

Hereinafter, the present invention will be described in greater detail with reference to the following examples. The following examples are for illustrative purposes only and are not intended to limit the scope of the invention.

### EXAMPLES

First, a method of synthesizing Compounds 1 to 4 will be described.

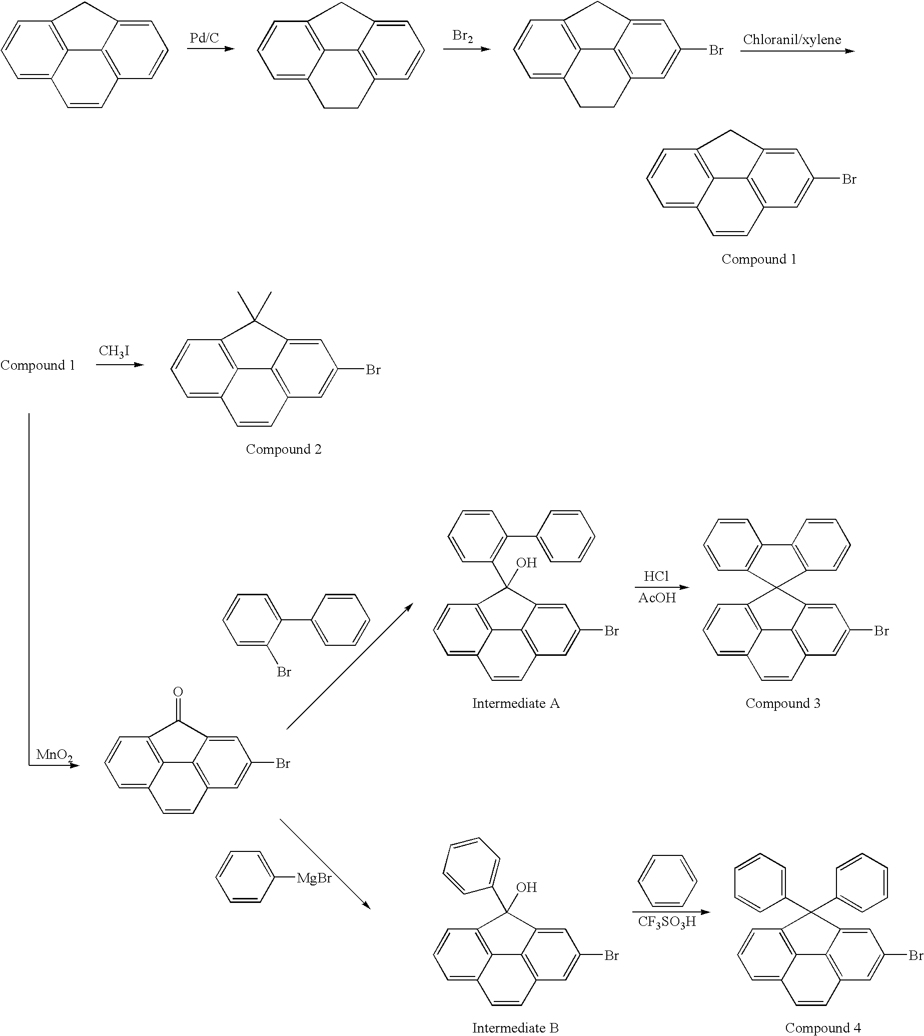

### Synthesis Example 1

### Synthesis of Compound 1

### 1) Synthesis of 8,9-dihydro-4H-cyclopenta[def]phenanthrene

4.75 g (25 mmol) of 4H-cyclopenta[def]phenanthrene was added to a Par reactor and 200 ml of ethanol was added thereto. 3.99 g of 5% Pd/C was added to the mixture and the mixture was reacted at 40 psi hydrogen pressure for 24 hours. When the reaction was completed, the reaction solution was filtered and the filtrate was concentrated in a reduced pressure to obtain 4.32 g of white solid 8,9-dihydro-4H-cyclopenta[def]phenanthrene (Yield: 90%).

### 2) Synthesis of 2-bromo-8,9-dihydro-4H-cyclopenta[def]phenanthrene

4.0 g (20.8 mmol) of 8,9-dihydro-4H-cyclopenta[def]phenanthrene was added to a 250 mW round-bottom flask (RBF) and dissolved in 100 mD of CCl₄. The mixture was cooled to 0° C. and 3.3 g (20.8 mmol) of Br₂was added to the reaction mixture. After the reaction was performed for 4 hours, a 10% NaSO₃ solution was added to the mixture and an organic layer was isolated. The isolated organic layer was concentrated in a reduced pressure and recrystallized using n-hexane to obtain 4.7 g of 2-bromo-8,9-dihydro-4H-cyclopenta[def]phenanthrene.

### 3) Synthesis of Compound 1 (2-bromo-4H-cyclopenta[def]phenanthrene)

4.45 g (16.4 mmol) of the prepared 2-bromo-8,9-dihydro-4H-cyclopenta[def]phenanthrene was added to a 250 ml round-bottom flask and dissolved in xylene, and 4.3 g of o-Chloranil was added thereto. The flask was reacted using an oil bath while heating and refluxing for 72 hours. After the reaction is completed, the reaction mixture was cooled and concentrated in a reduced pressure. The concentrated residue was separated using a silica gel column chromatography using n-hexane as a developing solvent to obtain 3.5 g of Compound 1 (Yield: 79%).

1H NMR (300 MHz, CDCl₃, δ): 7.98(2H, s), 7.79(2H, s), 7.73(2H, s), 6.94(dd, 1H), 4.28(2H, s)

### Synthesis Example 2

### Synthesis of Compound 2

2.6 g (9.66 mmol) of 2-bromo-4H-cyclopenta[def]phenanthrene, 20.8 g (61.6 mmol) of potassium t-butoxide, 20 ml of DMSO and 20 ml of HMPA were added to a 50 ml round-bottom flask using an injector. The mixture was stirred at room temperature for 50 minutes and cooled to 0° C. 3.75 ml (61.6 mmol) CH₃1 was added thereto at 0° C. using an injector, and the mixtue was stirred at 0° C. for 30 minutes. Then, 50 ml of water and 50 ml of methylene chloride were added to the mixture and an organic layer was separated using a silica gel column chromatography to obtain 3.6 g of Compound 2 (Yield: 80%).

1H NMR (300 MHz, CDCl₃, δ): 7.98(2H, s), 7.79(2H, s), 7.73(2H, s), 6.94(dd, 1H), 1.93(m, 6H).

### Synthesis Example 3

### Synthesis of Compound 3

### 1) Synthesis of 2-bromo-cyclopenta[def]phenanthrene-4-one

200 ml of benzene was added to a 250 ml round-bottom flask and 3.6 g (13.3 mmol) of Compound 1 was added thereto. 150 g of MnO₂was added thereto and the reaction mixture was reacted using an oil bath while heating and refluxing for 18 hours. When the reaction is completed, the reaction mixture was filtered to remove MnO₂ and the filtrate was sufficiently washed sequentially with chloroform, THF and methanol. The residual solution was concentrated in a reduced pressure and recrystallized using acetone to obtain 1.5 g of 2-bromo-cyclopenta[def]phenanthrene-4-one (Yield: 39%).

### 2) Synthesis of Intermediate A

0.68 g (2.95 mmol) of 2-bromo biphenyl was dissolved in 10 ml of anhydrous and the mixture was cooled to −78° C. Then, 3.5 ml of t-BuLi was gradually added thereto and the mixture was stirred for 1 hour, and a solution in which 1 g (3.53 mmol) of 2-bromo-cyclopenta[def]phenanthrene-4-one was dissolved in 5 ml of anhydrous THF 5 ml was added to the mixture for 30 minutes. When the reaction is completed, the reaction solution was concentrated in a reduced pressure and ethyl acetate and a NaCl solution were added to the residue to isolate an organic layer. The concentrated residue was separated using a silica gel column chromatography to obain 1 g of Intermediate A.

### 3) Synthesis of Compound 3

The obtained Intermediate A was dissolved in 30 ml of acetic acid, and the solution was cooled to 0° C. Then, 1 ml of hydrochloric acid was added thereto and the mixture was reacted for 2 hours. When the reaction was completed, a white solid formed during the reaction was filtered and washed with acetic acid and methanol to obtain 1.05 g of while solid Compoud 3 (Yield: 91%).

1H NMR(300 MHz, CDCl₃, δ): 7.22-7.26(m, 8H), 7.70(s, 2H), 7.80(s, 2H), 8.00(s, 2H)

### Synthesis Example 4

### Synthesis of Compound 4

### 1) Synthesis of Intermediate B

1.0 g (2.76 mmol) of 2-bromo-cyclopenta[def]phenanthrene-4-one was dissolved in a solvent including 30 ml of dried ether and 10 ml of THF. Phenylmmagnesium bromide (3.0 M in ether) was gradually added thereto in a nitrogen atmosphere and the mixture was refluxed for 3 hours. The reaction was completed by adding water thereto, and the pH of the mixture was adjusted to 3 to 4 usig 1 N-HCl solution, and then an organic material was subject to extraction using ethyl acetate. The organic layer was dried using anhydrous sodium sulfate and filtered. The filtrate was concentrated in a reduced pressure. The resultant solid was purified using a silica gel column chromatography to obtain 0.79 g of solid Intermediate B (Yield: 65%).

### 2) Synthesis of Compound 4

0.79 g (1.79 mmol) of the prepared Intermediate B was dissolved in 20 ml dried benzene. 0.48 ml (5.38 mmol, 3 equivalent weight) of trifluoromethanesulfonic acid was added thereto and the mixture was reacted at 80° C. for 2 hours. The mixture was diluted with water and subject to extraction to obtain an organic material. The organic layer was dried using anhydrous sodium sulfate and filtered. The filtrate was concentrated in a reduced pressure. The resultant solid was purified using a silica gel column chromatography and recrystallized using a EtOAc-Hex solvent to obtain 0.65 g of solid Compound 4 (Yield: 63%).

1H NMR(300 MHz, CDCl₃, δ):7.22-7.26(m, 10H), 7.70(s, 2H), 7.80(s, 3H), 8.00(s, 2H)

Hereinafter, a method of preparing Compounds 5 to 7 will be described.

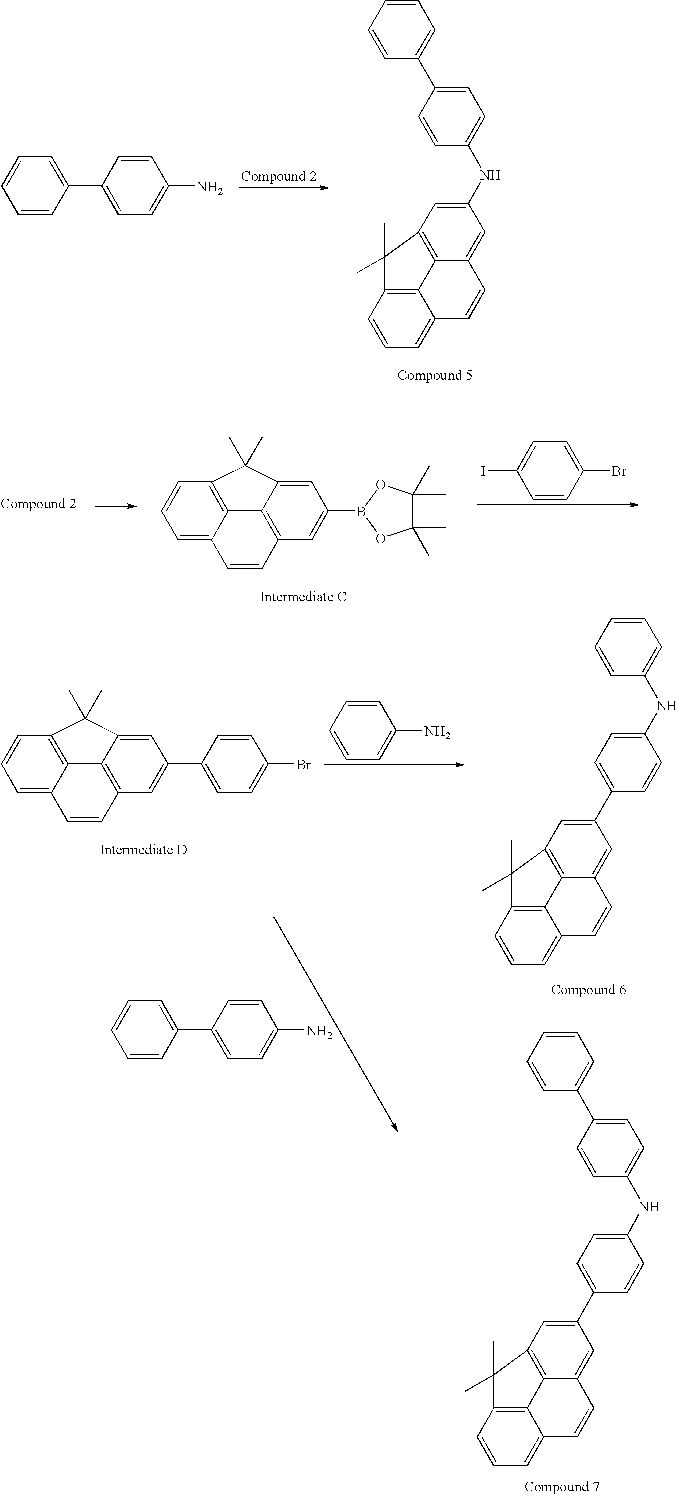

### Synthesis Example 5

### Synthesis of Compound 5

1.0 g (3.36 mmol) of Compound 2 synthesized in Synthesis Example 2, 2.28 g (13.46 mmol) of 4-bromobiphenyl, 0.31 g (3.696 mmol) of sodium tert-butoxide, 0.06 g of tris(dibenzylidine acetone)dipalladium(0) (Pd(dba)₂) and 0.07 g of tri(tert-butyl)phosphine were dissolved in 30 ml of toluene in a 50 ml round-bottom flask, and the mixture was refluxed for 12 hours to be reacted. When the reaction was completed, the mixture was cooled to room temperature, and 100 ml of distilled water was added thereto to extract an organic layer. The collected organic layer was dried using magnesium sulfate and filtered. The filtrate was concentrated and separated using a silica gel column chromatography. The resultant solution was concentrated 11 and further dried to obtain 1.1 g of solid Compound 5 (Yield: 85%). The obtained Compound 5 was identified by an atmospheric pressure chemical ionization (APCl) using a LCMS(SHIMADZU, LCMS-IT-TOF) ([M+H]+=386).

### Synthesis Example 6

### Synthesis of Compound 6

### 1) Synthesis of Intermediate C

1.0 g (3.36 mmol) of Compound 2 was dissolved in 10 ml of anhydrous THF in a 50 ml round-bottom flask and the mixture was cooled to −78° C. Then, 1.9 ml of 2.5 M n-butyl lithium was gradually added thereto and the mixture was stirred for 1 hour. Then, a solution in which 1.4 ml (6.72 mmol) of 2-isopropoxy-4,4,5,5-tetramethyl-1,3,2-dioxaborolane was dissolved in 5 ml of anhydrous THF was added to the mixture for 30 minutes. When the reaction is completed, the reaction solution was concentrated in a reduced pressure and ethyl acetate and a NaCl solution were added to the residue to isolate an organic layer. The organic layer was concentrated and separated using a silica gel column chromatography to obtain 0.95 g of Intermediate C (Yield: 82%).

### 2) Synthesis of Intermediate D

1.0 g (2.9 mmol) of the prepared Intermediate C, 1.6 g (5.8 mmol) of 4-iodobromobenzene, 0.23 g of tetrakis(triphenyl phosphine)palladium(0), 1 ml of 2 M potassium carbonate and 0.65 g of tetrabutyl ammonium bromide were added to a 100 mi round-bottom flask in an argon atmosphere, and 50 ml of THF and 20 ml of toluene were added thereto. The mixture was refluxed at 100° C. for 16 hours. When the reaction solution turned dark brown, water was added to the mixture and an organic material was extracted using ethyl acetate. The resultant organic layer was dried using anhydrous sodium sulfate and filtered to remove a solvent. The resultant was separated using a silica gel column chromatography by dissolving the resultant in a small amount of toluene to obtain 0.72 g of Intermediate D (Yield: 74%).

### 3) Synthesis of Compound 6

1.0 g (2.67 mmol) of the prepared Intermediate D, 0.25 g (2.67 mmol) of aniline, 0.28 g of sodium tert-butoxide, 0.05 g of tris(dibenzylidine acetone)dipalladium(0) (Pd(dba)₂), and 0.05 g of tri(tert-butyl)phosphine were dissolved in 30 ml of toluene in a 50 ml of round-bottom flask, and the reaction mixture was refluxed for 12 hours. When the reaction is completed, the reaction mixture was cooled to room temperature and an organic layer was extracted by adding 100 ml of distilled water. The collected organic layer was dried using magnesium sulfate and separated using a silica gel column chromatography. An eluate solution obtained therefrom was concentrated and dried to obtain 0.92 g of solid Compound 6. Compound 6 was identified by an atmospheric pressure chemical ionization (APCl) using a LCMS ([M+H]+=386).

Hereinafter, a method of synthesizing Compounds 8 and 9 will be described.

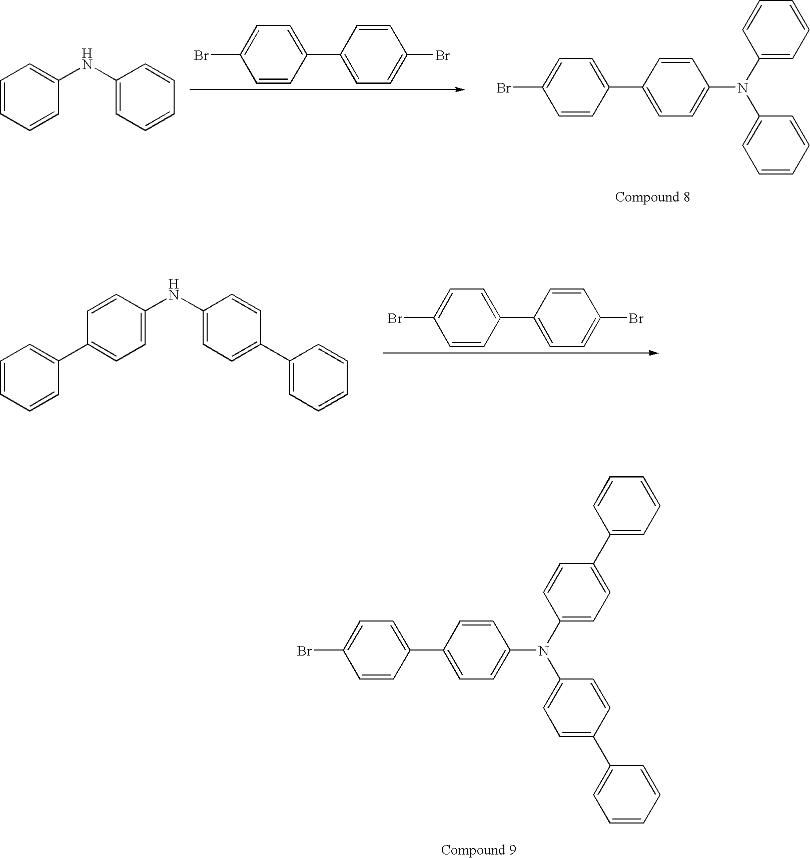

### Synthesis Example 7

### Synthesis of Compound 7

1.94 g (6.22 mmol) of 4,4-dibromobiphenyl, 0.53 g (3.11 mmol) of biphenyl amine, 0.45 g of sodium tert-butoxide, 0.03 g of tris(dibenzylidine acetone)dipalladium(0) (Pd(dba)₂) and 0.01 g of tri(tert-butyl)phosphine were dissolved in 30 ml of toluene in a 50 ml round-bottom flask and the mixture was refluxed for 12 hours. When the reaction was completed, the mixture was cooled to room temperature, and 100 ml of distilled water was added thereto to extract an organic layer. The collected organic layer was dried using magnesium sulfate and filtered. The filtrate was concentrated and separated using a silica gel column chromatography. An eluate solution obtained therefrom was concentrated and dried to obtain 0.6 g of solid Compound 7 (Yield: 48%). Compound 7 was identified by an atmospheric pressure chemical ionization (APCl) using a LCMS ([M+H]+=400).

### Synthesis Example 9

### Synthesis of Compound 9

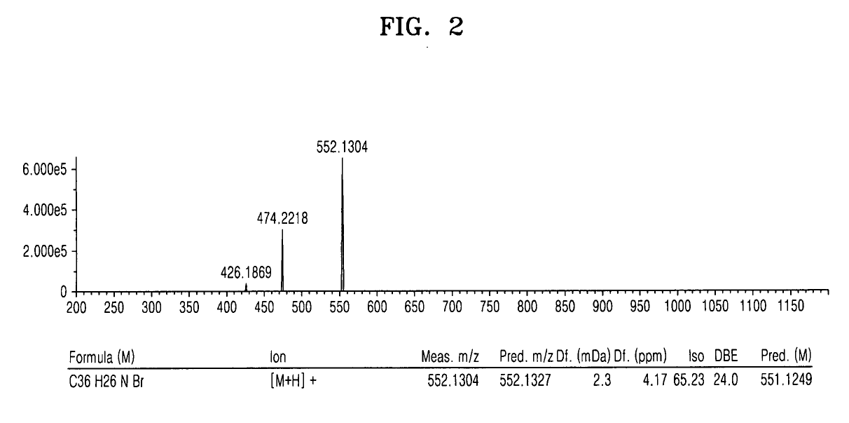

1.46 g (4.67 mmol) of 4,4-dibromobiphenyl, 1.0 g (3.11 mmol) of bisbiphenyl amine, 0.3 g of sodium tert-butoxide, 0.05 g of tris(dibenzylidine acetone)dipalladium(0) (Pd(dba)₂) and 0.05 g of tri(tert-butyl)phosphine were dissolved in 30 ml of toluene in a 50 ml round-bottom flask, and the reaction mixture was refluxed for 12 hours. When the reaction is completed, the reaction mixture was cooled to room temperature and an organic layer was extracted by adding 100 ml of distilled water. The collected organic layer was dried using magnesium sulfate and filtered. The filtrate was separated using a silica gel column chromatography. An eluate solution obtained therefrom was concentrated and dried to obtain 0.42 g of solid Compound 9. Compound 9 was identified by an atmospheric pressure chemical ionization (APCl) using a LCMS ([M+H]+=552). FIG. 2 is a graph illustrating liquid chromatography-mass spectrometry (LC-MS) results of a compound prepared according to Example 1.

Hereinafter, a method of preparing a cyclopentaphenanthrene-based compound according to an embodiment of the present invention will be described.

### Example 1

### Synthesis of a Compound Represented by Formula 11

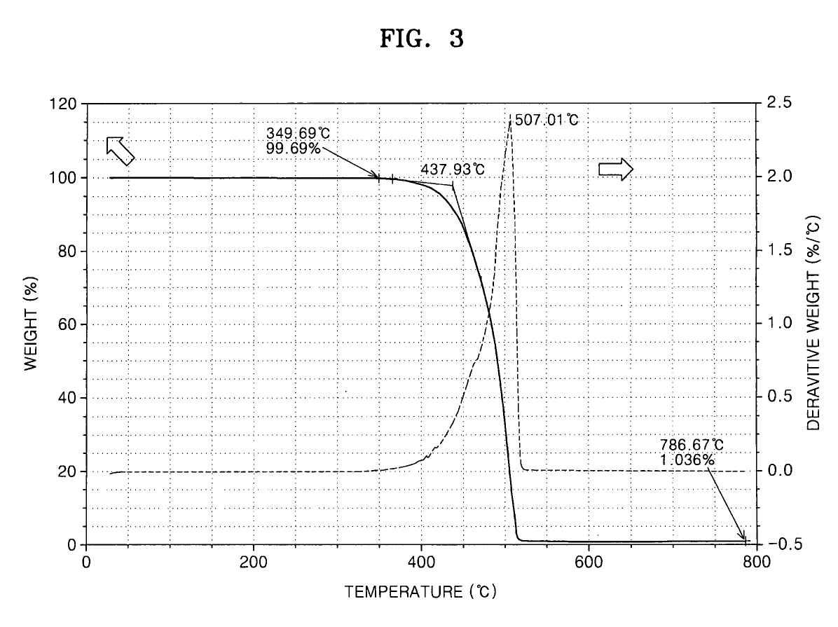

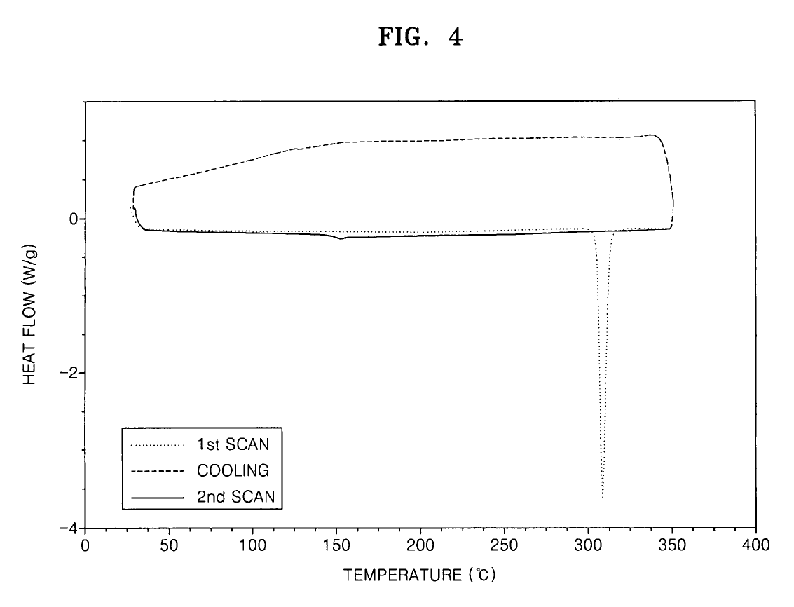

1.0 g (2.97 mmol) of N,N-diphenyl benzidine, 2.2 g (7.43 mmol) of Compound 2 obtained according to Synthesis Example 2, 0.8 g of sodium tert-butoxide, 0.3 g of tris(dibenzylidine acetone)dipalladium(0) (Pd(dba)₂) and 0.07 g of tri(tert-butyl)phosphine were dissolved in 50 ml of toluene in a 50 ml of round-bottom flask, and the mixture was refluxed for 12 hours to be reacted. When the reaction was completed, the mixture was cooled to room temperature, and 100 ml of distilled water was added thereto to extract an organic layer. The collected organic layer was dried using magnesium sulfat, concentrated and separated using a silica gel column chromatography. An eluate solution obtained therefrom was concentrated and further dried to obtain 0.86 g of solid compound represented by Formula 11 (Yield: 75%). The obtained compound of Formula 11 was identified by an atmospheric pressure chemical ionization (APCl) using a LCMS (SHIMADZU, LCMS-IT-TOF) ([M+H]+=769). FIG. 2 is a graph illustrating LCMS results of a compound prepared according to Example 1. In addition, thermal analysis of the compound of Formula 11 was performed using a thermo gravimetric anaysis (TGA) in a nitrogen atmosphere at a temperature in the range of room temperature to 600° C. at 100° C./min in a disposable Al pan and a differential scanning calorimetry (DSC) at a temperature in the range of room temperature to 4000° C. in a disposable Al pan. As a result, Td was 438° C. and Tg was 149° C. FIG. 3 is a graph illustrating thermogravimetric analysis (TGA) results of a compound prepared according to Example 1 and FIG. 4 is a graph illustrating differential scanning calorimetry (DSC) of a compound prepared according to Example 1.

### Example 2

### Synthesis of a Compound Represented by Formula 20

3.08 g (8.01 mmol) of Compound 5, 1.0 g (3.2 mmol) of 4,4-dibromobiphenyl, 0.9 g of sodium tert-butoxide, 0.4 g of tris(dibenzylidine acetone)dipalladium(0) (Pd(dba)₂) and 0.1 g of tri(tert-butyl)phosphine were dissolved in 50 ml toluene in a 50 ml round-bottom flask and the mixture was refluxed for 12 hours to be reacted. When the reaction was completed, the mixture was cooled to room temperature, and 100 ml of distilled water was added thereto to extract an organic layer. The collected organic layer was dried using magnesium sulfate, concentrated and separated using a silica gel column chromatography. An eluate solution obtained therefrom was concentrated and further dried to obtain 2.6 g of a solid compound represented by Formula 20 (Yield: 88%). The obtained compound of Formula 20 was identified by an atmospheric pressure chemical ionization (APCl) using a LCMS(SHIMADZU, LCMS-IT-TOF) ([M+H]+=921).

### Example 3

### Synthesis of a Compound Represented by Formula 21

1.0 g (2.59 mmol) of Compound 5, 1.8 g (3.24 mmol) of Compound 9, 0.8 g of sodium tert-butoxide, 0.08 g of tris(dibenzylidine acetone)dipalladium(0) (Pd(dba)₂) and 0.05 g of tri(tert-butyl)phosphine were dissolved in 50 ml of toluene in a 50 ml of round-bottom flask, and the mixture was refluxed for 12 hours to be reacted. When the reaction was completed, the mixture was cooled to room temperature, and 100 ml of distilled water was added thereto to extract an organic layer. The collected organic layer was dried using magnesium sulfate, concentrated and separated using a silica gel column chromatography. An eluate solution obtained therefrom was concentrated and further dried to obtain 1.1 g of a compound represented by Formula 21 (Yield: 70%). The obtained compound of Formula 21 was identified by an atmospheric pressure chemical ionization (APCl) using a LCMS (SHIMADZU, LCMS-IT-TOF) ([M+H]+=857).

### Example 4

### Synthesis of a Compound Represented by Formula 29

1.0 g (2.59 mmol) of Compound 6, 1.03 g (2.59 mmol) of Compound 8, 0.7 g of sodium tert-butoxide, 0.1 g of tris(dibenzylidine acetone)dipalladium(0) (Pd(dba)₂) and 0.03 g of tri(tert-butyl)phosphine were dissolved in 50 ml of toluene in a 50 ml of round-bottom flask, and the mixture was refluxed for 12 hours to be reacted. When the reaction was completed, the mixture was cooled to room temperature, and 100 ml of distilled water was added thereto to extract an organic layer. The collected organic layer was dried using magnesium sulfate, concentrated and separated using a silica gel column chromatography. An eluate solution obtained therefrom was concentrated and further dried to obtain 1.3 g of a solid compound represented by Formula 29 (Yield: 71%). The obtained compound of Formula 29 was identified by an atmospheric pressure chemical ionization (APCl) using a LCMS (SHIMADZU, LCMS-IT-TOF) ([M+H]+=704).

### Example 5

### Synthesis of a Compound Represented by Formula 30

A compound represented by Formula 30 was prepared in the same manner as in Example 2 except that Compound 6 was used instead of Compoun 5.

### Example 6

### Synthesis of a Compound Represented by Formula 32

A compound represented by Formula 32 was prepared in the same manner as in Example 3 except that Compound 7 was used instead of Compoun 5.

### Example 7

### Synthesis of a Compound Represented by Formula 34

A compound represented by Formula 34 was prepared in the same manner as in Example 1 except that Compound 3 was used instead of Compoun 2.

### Example 8

### Synthesis of a Compound Represented by Formula 43

A compound represented by Formula 43 was prepared in the same manner as in Example 1 except that Compound 4 was used instead of Compoun 2.

### Example 9

### Synthesis of a Compound Represented by Formula 58

A compound represented by Formula 58 was prepared in the same manner as in Example 2 except that 2,7-dibromo-9,9-dimethylfluorene was used instead of 4,4-dibromobiphenyl.

Hereinafter, manufacturing and evaluating organic light emitting devices according to embodiments of the present invention will be described.

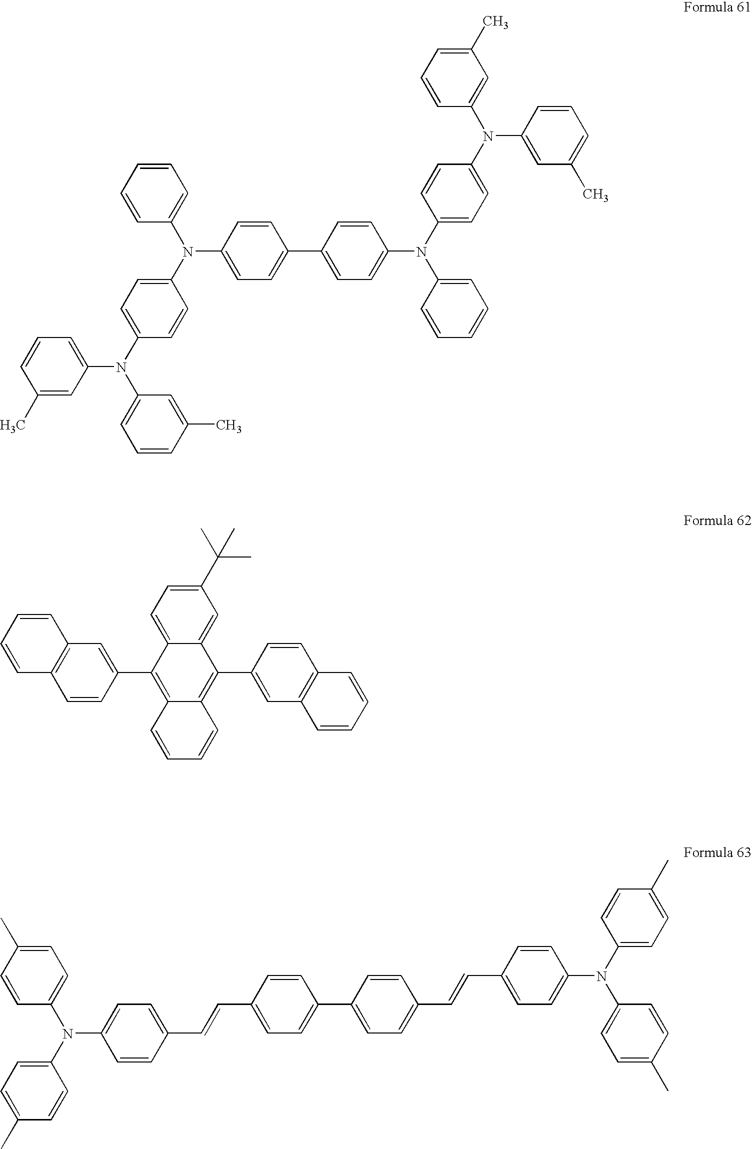

### Example 10

### Manufacturing and Evaluating Organic Light Emitting Devices

An organic light emitting device having the following structure was manufactured using a compound represented by Formula 61 as a hole injection layer, a compound represented by Formula 11 prepared in Example 1 as a hole transport layer, a compound represented by Formula 62 as a host of an emitting layer and a compound represented by Formula 63 as a dopant of an emitting layer: ITO/compound of Formula 61 (600 Å)/compound of Formula 11 (300 Å)/compound of Formula 62:compound of Formula 63(300 Å)/Alq3(40 Å)/LiF(10 Å)/Al(2000 Å).

15 Ω/cm² (1000 Å) ITO glass substrate was cut to a size of 50 mm×50 mm×0.7 mm, microwave washed with acetone isopropyl alcohol for 15 minutes, microwave washed with pure water for 15 minutes, and washed with UV ozone for 30 minutes. The compound of Formula 61 was vacuum deposited on the substrate to form a hole injection layer and the compound of Formula 11 was vacuum deposited thereon to form a hole transport layer. Then, compounds of Formulas 62 and 63 were vacuum deposited in a weight ratio of 100:5 to form an emitting layer. Then, Alq3 was vacuum deposited on the emitting layer to form an electron transport layer with a thickness of 40 Å. LiF was vacuum deposited on the electron transport layer to form an electron injection layer with a thickness of 10 Å, and then Al was vacuum deposited on the electron injection layer to form a cathode with a thickness of 2000 Å. As a result, an organic light emitting device illustrated in FIG. 1A was manufactured.

The obtained organic light emitting device had 14,000 cd/m² of blue light emitting at 6.0 V and efficiency of 6.97 cd/A. Driving voltage, emitting efficiency and brightness half-life of the organic light emitting device were measured when the organic light emitting device is driven at a constant current of 30 mA/cm², and the results are shown in Table 1.

### Example 11 to 18

### Manufacturing and Evaluating Organic Light Emitting Devices

Organic light emitting devices were manufactured in the same manner as in Example 10 except that compounds prepared according to Examples 2 to 7 were used instead of the compound of Formula 11 prepared according to Example 1 as a hole transport layer. Driving voltage, emitting efficiency and brightness half-life of the organic light emitting device were measured when the organic light emitting device is driven at a constant current of 30 mA/cm², and the results are shown in Table 1.

### Comparative Example 1

### Manufacturing and Evaluating Organic Light Emitting Devices

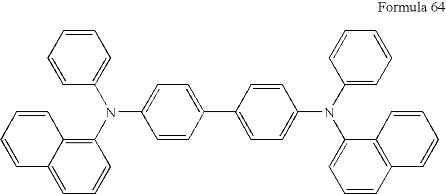

An organic light emitting device was manufactured in the same manner as in Example 1 except that a compound of Formula 64 was used instead of the compound of Formula 11 prepared according to Example 1 as a hole transport layer. Driving voltage, emitting efficiency and brightness half-life of the organic light emitting device were measured when the organic light emitting device is driven at a constant current of 30 mA/cm², and the results are shown in Table 1.

TABLE 1

| | | | Emitting | |
|---|---|---|---|---|
| | | Driving | efficiency | Brightness |
| | Compound used | voltage (V) | (cd/A) | half-life (hr) |
| Example 10 | Example 1 | 7.2 | 6.85 | 1130 |
| | (Formula 11) | | | |
| Example 11 | Example 2 | 7.1 | 6.68 | 1240 |
| | (Formula 20) | | | |
| Example 12 | Example 3 | 7.1 | 6.53 | 1210 |
| | (Formula 21) | | | |
| Example 13 | Example 4 | 7.2 | 6.3 | 950 |
| | (Formula 29) | | | |
| Example 14 | Example 5 | 7.2 | 6.57 | 1200 |
| | (Formula 30) | | | |
| Example 15 | Example 6 | 7.1 | 6.70 | 1330 |
| | (Formula 32) | | | |
| Comparative | Comparative | 7.3 | 6.2 | 900 |
| Example 1 | Example 1 | | | |
| | (Formula 64) | | | |

As shown in Table 1, the organic light emitting devices prepared according to Examples 10 to 16 have low driving voltage and improved emitting efficiency compared to the organic light emitting device prepared according to Comparative Example 1. Thus, it can be seen that the cyclopentaphenanthrene-based compound according to embodiments of the present invention has excellent hole transporting capability. In addition, the organic light emitting devices manufactured according to Examples 10 to 16 have increased lifetime compared to the organic ligher emitting device accoding to Comparative Example 1. Those properties of the organic light emitting device are related to high glass transition temperature and high thermal stability of the cyclopentaphenanthrene-based compound according to embodiments of the present invention.

According to the present invention, a cyclopentaphenanthrene-based compound represented by Formula 1 has high solubility in a solvent in the formation of an organic layer, high thermal stability and excellent charge transporting properties. In addition, an organic light emitting device according to the present invention has low driving voltage, high efficiency and long lifetime.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An organic compound represented by Formula 1 below: 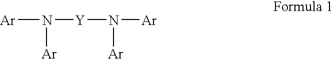
wherein Y, as a bivalent linking group, is a substituted or unsubstituted C6-C30 arylene group or a substituted or unsubstituted C2-C30 heteroarylene group;
at least one of Ars is a substituent represented by Formula 2, and the others which are identical to or different from each other are a substituted or unsubstituted C6-C30 aryl group: 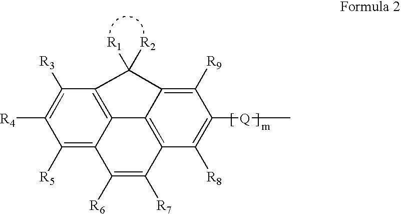
wherein R₁ and R₂ are each independently selected from the group consisting of a hydrogen atom, a halogen atom, a cyano group, a hydroxyl group, a substituted or unsubstituted C1-C20 alkyl group, a substituted or unsubstituted C3-C20 cycloalkyl group, a substituted or unsubstituted C5-C30 heterocycloalkyl group, a substituted or unsubstituted C1-C20 alkoxy group, a substituted or unsubstituted C6-C30 aryl group, a substituted or unsubstituted C6-C30 aralkyl group and a substituted or unsubstituted C2-C30 heteroaryl group; or
R₁ and R₂ are linked to form one selected from the group consisting of a substituted or unsubstituted C3-C20 aliphatic ring, a substituted or unsubstituted C5-C30 heteroaliphatic ring, a substituted or unsubstituted C6-C30 aromatic ring and a substituted or unsubstituted C2-C30 heteroaromatic ring;
R₃ to R₉ are each independently selected from the group consisting of a hydrogen atom, a halogen atom, a cyano group, a hydroxyl group, a substituted or unsubstituted C1-C20 alkyl group, a substituted or unsubstituted C3-C20 cycloalkyl group, a substituted or unsubstituted C5-C30 heterocycloalkyl group, a substituted or unsubstituted C1-C20 alkoxy group, a substituted or unsubstituted C6-C30 aryl group, a substituted or unsubstituted C6-C30 aralkyl group and a substituted or unsubstituted C2-C30 heteroaryl group;
m is an integer of 0 to 2; and
Q is a bivalent substituent represented by any one of compounds below:

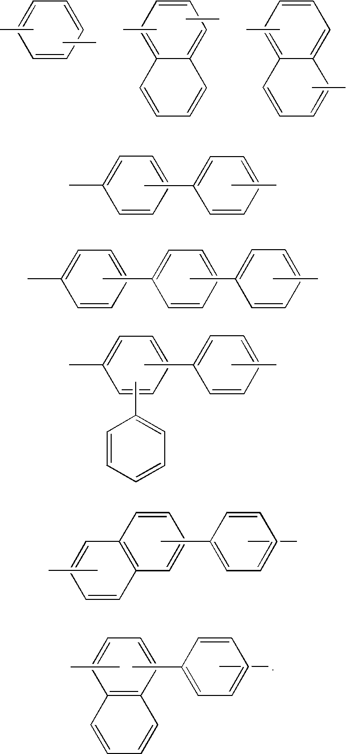

2. The organic compound of claim 1, wherein when R₁ and R₂ are linked to form a ring, the compound represented by Formula 2 is represented by any one of Formulae 3 to 6 below: 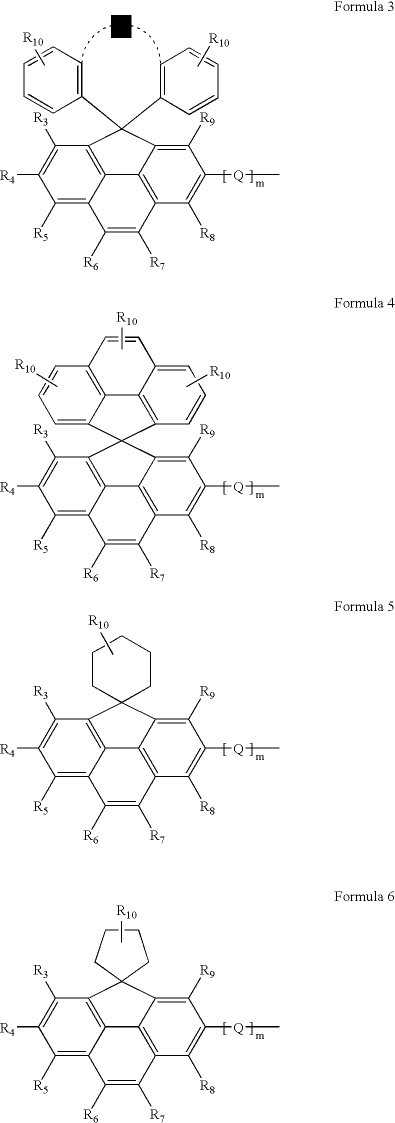
wherein R₁₀ is selected from the group consisting of a hydrogen atom, a halogen atom, a cyano group, a hydroxyl group, a substituted or unsubstituted C1-C20 alkyl group, a substituted or unsubstituted C3-C20 cycloalkyl group, a substituted or unsubstituted C5-C30 heterocycloalkyl group, a substituted or unsubstituted C1-C20 alkoxy group, a substituted or unsubstituted C6-C30 aryl group, a substituted or unsubstituted C6-C30 aralkyl group and a substituted or unsubstituted C2-C30 heteroaryl group;
A is an oxygen atom, a sulfur atom or —(CH₂)ₚ—, with the proviso that p is an integer of 1 to 5; and
R₃ to R₉ are each independently selected from the group consisting of a hydrogen atom, a halogen atom, a cyano group, a hydroxyl group, a substituted or unsubstituted C1-C20 alkyl group, a substituted or unsubstituted C3-C20 cycloalkyl group, a substituted or unsubstituted C5-C30 heterocycloalkyl group, a substituted or unsubstituted C1-C20 alkoxy group, a substituted or unsubstituted C6-C30 aryl group, a substituted or unsubstituted C6-C30 aralkyl group and a substituted or unsubstituted C2-C30 heteroaryl group;
M is an integer of 0 to 2; and
Q is a bivalent substituent represented by any one of compounds below:

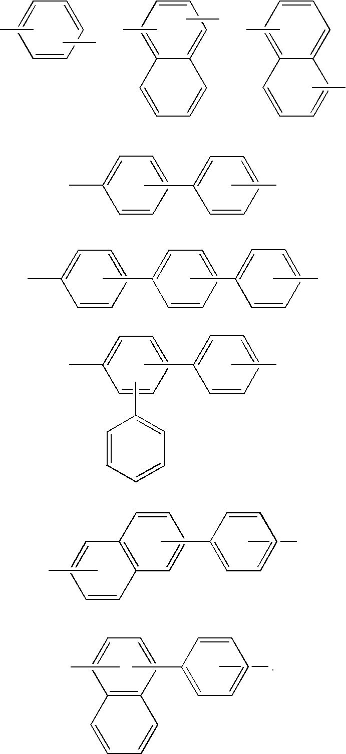

3. The organic compound of claim 1, wherein Y is a bivalent linking group represented by any one of the compounds represented by formulae below: 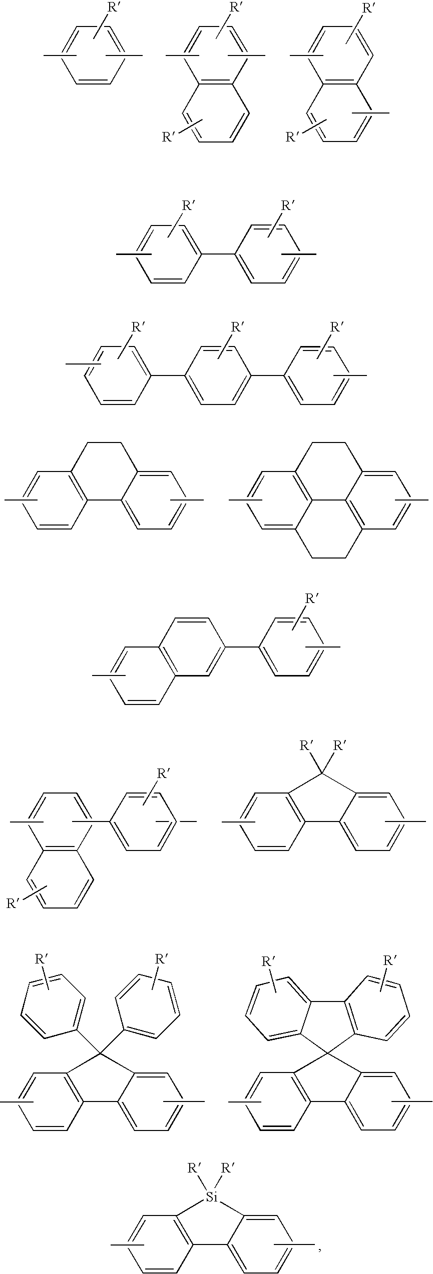
wherein R′ is selected from the group consisting of a hydrogen atom, a halogen atom, a cyano group, a hydroxyl group, a substituted or unsubstituted C1-C20 alkyl group, a substituted or unsubstituted C3-C20 cycloalkyl group, a substituted or unsubstituted C5-C30 heterocycloalkyl group and a substituted or unsubstituted C1-C20 alkoxy group.

4. The organic compound of claim 1, wherein the compound represented by Formula 2 is represented by any one of the compounds represented by Formulae 7 to 9: 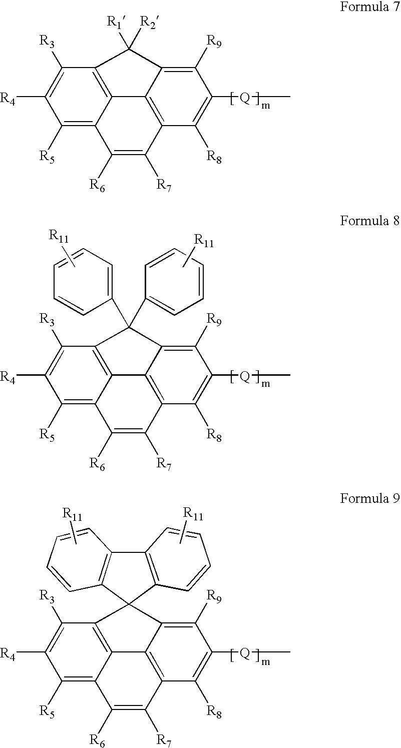
wherein R₁′, R₂′ and R₁₁ are each independently selected from the group consisting of a hydrogen atom, a halogen atom, a cyano group, a hydroxyl group, a substituted or unsubstituted C1-C20 alkyl group, a substituted or unsubstituted C3-C20 cycloalkyl group, a substituted or unsubstituted C5-C30 heterocycloalkyl group, a substituted or unsubstituted C1-C20 alkoxy group, a substituted or unsubstituted C6-C30 aryl group, a substituted or unsubstituted C6-C30 aralkyl group and a substituted or unsubstituted C2-C30 heteroaryl group; and
R₃ to R₉ are each independently selected from the group consisting of a hydrogen atom, a halogen atom, a cyano group, a hydroxyl group, a substituted or unsubstituted C1-C20 alkyl group, a substituted or unsubstituted C3-C20 cycloalkyl group, a substituted or unsubstituted C5-C30 heterocycloalkyl group, a substituted or unsubstituted C1-C20 alkoxy group, a substituted or unsubstituted C6-C30 aryl group, a substituted or unsubstituted C6-C30 aralkyl group and a substituted or unsubstituted C2-C30 heteroaryl group;
m is an integer of 0 to 2; and
Q is a bivalent substituent represented by any one of compounds below:

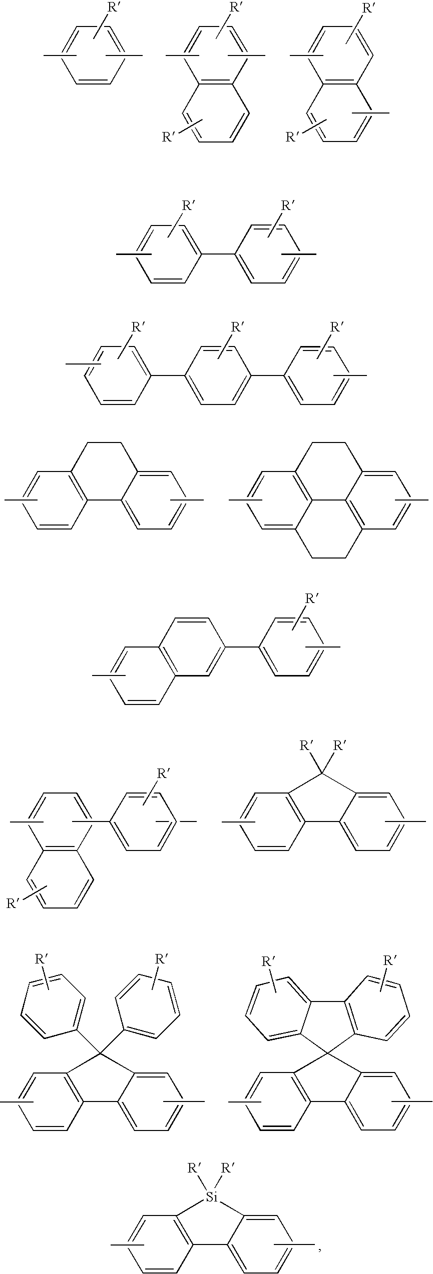

5. The organic compound of claim 1, wherein at least one of Ars is a substituent represented by Formula 2, and the others which are identical to or different from each other are any one of the compounds represented by formulae below: 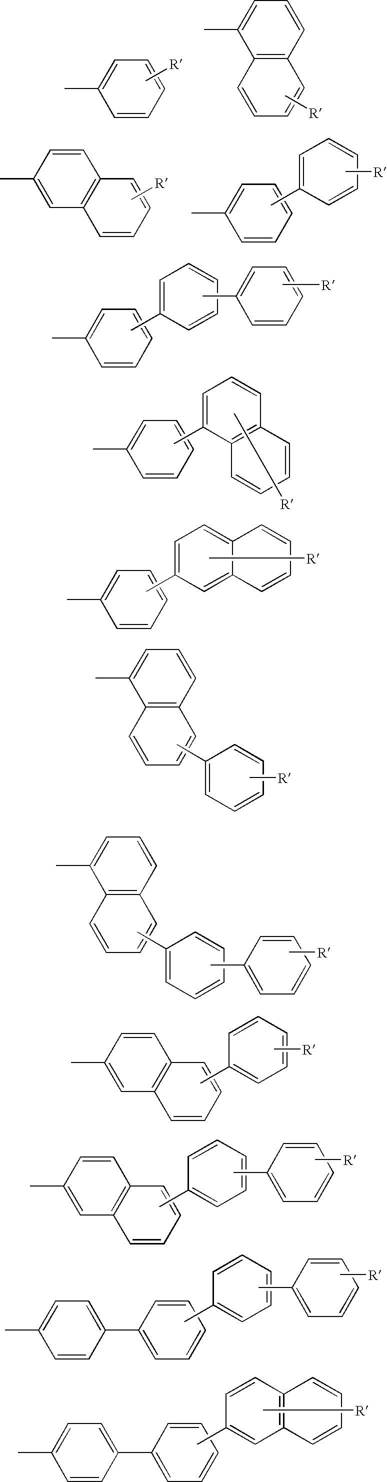 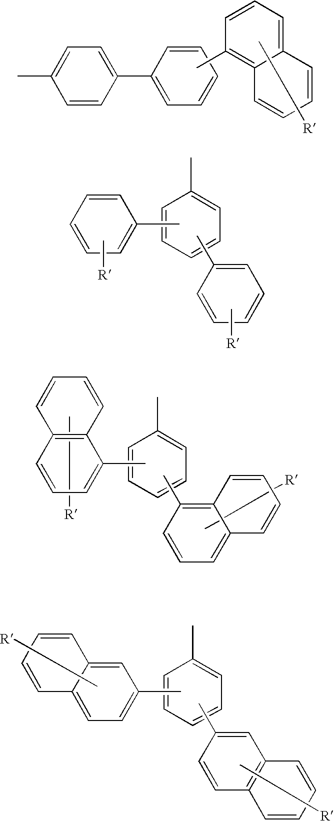
wherein R′ is selected from the group consisting of a hydrogen atom, a halogen atom, a cyano group, a hydroxyl group, a substituted or unsubstituted C1-C20 alkyl group, a substituted or unsubstituted C3-C20 cycloalkyl group, a substituted or unsubstituted C5-C30 heterocycloalkyl group and a substituted or unsubstituted C1-C20 alkoxy group.

6. The organic compound of claim 1, wherein m is an integer of 0 or 1.

7. An organic light emitting device comprising:
a first electrode first electrode;
a second electrode; and
at least one organic layer between the first electrode and the second electrode,
wherein the organic layer comprises a compound according to claim 1.

8. An organic light emitting device comprising:
a first electrode first electrode;
a second electrode; and
at least one organic layer between the first electrode and the second electrode,
wherein the organic layer comprises a compound according to claim 2.

9. An organic light emitting device comprising:
a first electrode first electrode;
a second electrode; and
at least one organic layer between the first electrode and the second electrode,
wherein the organic layer comprises a compound according to claim 3.

10. An organic light emitting device comprising:
a first electrode first electrode;
a second electrode; and
at least one organic layer between the first electrode and the second electrode,
wherein the organic layer comprises a compound according to claim 4.

11. An organic light emitting device comprising:
a first electrode first electrode;
a second electrode; and
at least one organic layer between the first electrode and the second electrode,
wherein the organic layer comprises a compound according to claim 5.

12. An organic light emitting device comprising:
a first electrode first electrode;
a second electrode; and
at least one organic layer between the first electrode and the second electrode,
wherein the organic layer comprises a compound according to claim 6.

13. The organic light emitting device of claim 7, wherein the organic layer is an emitting layer, a hole injection layer or a hole transport layer.

14. The organic light emitting device of claim 7, wherein the organic layer is a hole transport layer.

15. The organic light emitting device of claim 7, further comprising at least one layer selected from the group consisting of an emitting layer, a hole injection layer, a hole transport layer, an electron blocking layer, a hole blocking layer, an electron transport layer and an electron injection layer between the first electrode and the second electrode.

16. The organic light emitting device of claim 13, further comprising at least one layer selected from the group consisting of an emitting layer, a hole injection layer, a hole transport layer, an electron blocking layer, a hole blocking layer, an electron transport layer and an electron injection layer between the first electrode and the second electrode.

17. The organic light emitting device of claim 14, further comprising at least one layer selected from the group consisting of an emitting layer, a hole injection layer, a hole transport layer, an electron blocking layer, a hole blocking layer, an electron transport layer and an electron injection layer between the first electrode and the second electrode.
